(19) United States
(12) Patent Application Publication
(10) Pub. No.: US 2026/0284118 A1
(43) Pub. Date: Sep. 24, 2026

(54) IMMUNE THERAPY AND CANCER

(71) Applicant: THE BOARD OF TRUSTEES OF THE UNIVERSITY OF ILLINOIS, Urbana, IL (US)

(72) Inventors: Erik Russell NELSON, Champaign, IL (US); Shruti BENDRE, Champaign, IL (US); Andrew M. SMITH, Savoy, IL (US)

(21) Appl. No.: 19/574,620

(22) Filed: Mar. 23, 2026

Related U.S. Application Data
(60) Provisional application No. 63/775,567, filed on Mar. 21, 2025.

(51) Int. Cl. A61K 35/28; A61K 38/17; A61P 35/00

(52) U.S. Cl. CPC A61K 35/28; A61K 38/177; A61K 38/1793; A61P 35/00

(57) ABSTRACT

The disclosure provides for methods of using ApoA-I mimetic polypeptides to treat cancers and/or active myeloid cell by administering to a subject an ApoA-I mimetic polypeptide. Other embodiments provide cell therapy methods using engineered myeloid cells overexpressing the ABCA1 protein.

### RELATED APPLICATIONS

This application claims priority under 35 U.S.C. § 119(e) to U.S. Provisional Patent Application No. 63/775,567, filed Mar. 21, 2025, which is incorporated herein by reference.

### GOVERNMENT SUPPORT

This invention was made with government support under W81XWH2110177 awarded by the Defense Health Agency, Medical Research and Development Branch (DHA/MRDB). The United States Government has certain rights in the invention.

### REFERENCE TO SEQUENCE LISTING

The present application includes a Sequence Listing in electronic format as an xml file titled “500.167US1_SL” which was created on Mar. 20, 2026, and has a size of 24,576 bytes. The contents of xml file 500.167US1_SL are incorporated herein by reference.

### BACKGROUND OF THE INVENTION

Immune therapies such as chimeric antigen receptor T-cell (CAR T cells) and immune checkpoint blockade (ICB) have had enormous impact on the clinical treatment of cancer, and in some cases even offering curative therapy. However, CAR T cells require a cancer specific or enriched epitope to target, restricting their broad applicability. Furthermore, ICB has limited efficacy in many solid tumors, including metastatic breast cancer. Pembrolizumab (anti programmed cell death protein 1; αPD1) in combination with nab-paclitaxel for patients with advanced triple-negative breast cancer (TNBC) positive for the PD1 ligand (PD-L1) improves pathologic complete response and event-free survival. However, the vast majority of patients do not respond. Although αPD1 in the neoadjuvant setting delays progression, it is not curative. Furthermore, addition of αPD-L1 (Atezolizumab) to standard of care postoperative chemotherapy did not improve recurrence rates. Given that metastatic disease accounts for the majority of mortality associated with breast cancer, 1 in 8 women will be diagnosed with invasive breast cancer within their lifetime, and breast cancer being the second leading cause of cancer-related deaths in women, it is of paramount importance to probe mechanisms that may improve the efficacy of ICB.

Cholesterol and regulators of its homeostasis have emerged as important modulators of tumor immunology. Elevated circulating cholesterol has been associated with breast cancer recurrence. On the other hand, use of cholesterol lowering medication (statins) is associated with increased time to breast cancer recurrence. This association is also true for TNBC patients taking statins, demonstrating improved overall survival and breast cancer specific survival. However, statins have a high first-pass metabolism, allowing for tumors to escape and produce their own cholesterol. Thus, therapeutics targeting downstream metabolism or actions of cholesterol are required to truly harness the clinical associations between cholesterol and progression.

Cholesterol has been shown to influence several different immune cell types. Since ICB already targets T cells, we have focused on myeloid immune cells, which have been strongly implicated in tumor progression, resistance to conventional therapy, and resistance to ICB. However, myeloid cells are required for antigen presentation, T cell support and effective anti-tumor responses. Therapeutic strategies where they are removed or inhibited have failed. As such, what is needed are new methods to efficiently and effectively activate myeloid cells. The present invention satisfies these needs.

### SUMMARY OF THE INVENTION

The cholesterol homeostatic pathway involves several proteins, offering potentially new therapeutic targets. Here, we describe how the cholesterol efflux protein, ABCA1, alters several aspects of macrophage function. These functions have important implications within the tumor microenvironment. Correlations of ABCA1 and markers for myeloid cells and T cells in human breast tumors corroborate our preclinical findings. Collectively, our data suggest that ABCA1 represents a target to re-educate myeloid cells for the treatment of breast and other solid tumors.

Accordingly, in some embodiments, a method of cell therapy comprises the steps of i) transforming a population of myeloid cells in vitro with a polynucleotide encoding an ABCA1 gene to form a population of engineered myeloid cells; ii) expanding the population of engineered myeloid cells; and iii) administering an effective amount of the expanded population of engineered myeloid cells to a subject having or suspected of having a cancer, thereby treating the cancer when present.

In other embodiments, a method of treating cancer in a subject comprising administering to the subject a therapeutically effective amount of an ApoA-I mimetic polypeptide, thereby treating the cancer, wherein the ApoA-I mimetic polypeptide comprises an amino acid sequence that is at least about 85% identical to: DWLKAFYDKVAEKLKEAFPDWAKAAYDKAAEKAKEAA (SEQ ID NO: 1); PVLDLFRELLNELLEALKQKLK (SEQ ID NO: 2); DWFKAFYDKVAEKFKEAF (SEQ ID NO: 3); or ^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F (SEQ ID NO: 4); wherein superscript “D” indicates a D-amino acid, optionally, an acetyl moiety (Ac) is covalently bonded to an N-terminus nitrogen of the ApoA-I mimetic polypeptide, and optionally, an amine group is covalently bonded to the carbon of the C-terminus of the ApoA-I mimetic polypeptide.

In some embodiments, the ApoA-I mimetic polypeptide comprises the amino acid sequence: DWLKAFYDKVAEKLKEAFPDWAKAAYDKAAEKAKEAA (SEQ ID NO: 1); PVLDLFRELLNELLEALKQKLK (SEQ ID NO: 2); Ac-DWFKAFYDKVAEKFKEAF (SEQ ID NO: 3); or Ac-^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F (SEQ ID NO: 4).

In some embodiments, the ApoA-I mimetic polypeptide may include an acetyl moiety (Ac) covalently bonded to an N-terminus nitrogen and an amine group covalently bonded to C-terminus carbon of the ApoA-I mimetic polypeptide. For example, the ApoA-I mimetic polypeptide of SEQ ID NO: 1 can be i) DWLKAFYDKVAEKLKEAFPDWAK AAYDKAAEKAKEAA (SEQ ID NO: 1), ii) Ac-DWLKAFYDKVAEKLKEAFPDW AKAAYDKAAEKAKEAA (SEQ ID NO: 1), iii) ADWLKAFYDKVAEKLKEAFPDW AKAAYDKAAEKAKEAA-NH₂ (SEQ ID NO: 1), or iv) Ac-DWLKAFYDKVAEKLKEAFPDW AKAAYDKAAEKAKEAA-NH₂ (SEQ ID NO: 1). The ApoA-I mimetic polypeptide of SEQ ID NO: 2 can be i) PVLDLFRELLNELLEALKQKLK (SEQ ID NO: 2), ii) Ac-PVLDLFRELLNELLEALKQKLK (SEQ ID NO: 2), iii) PVLDLFRELLNELLEALKQKLK-NH₂ (SEQ ID NO: 2), or iv) Ac-PVLDLFRELLNELLEALKQKLK-NH₂. The ApoA-I mimetic polypeptide of SEQ ID NO: 3 can be i) DWFKAFYDKVAEKFKEAF (SEQ ID NO: 3), ii) Ac-DWFKAFYDKVAEKFKEAF (SEQ ID NO: 3), iii) DWFKAFYDKVAEKFKEAF-NH₂ (SEQ ID NO: 3), or iv) Ac-DWFKAFYDKVAEKFKEAF-NH₂ (SEQ ID NO: 3). The ApoA-I mimetic polypeptide of SEQ ID NO: 4 can be i) ^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F (SEQ ID NO: 4), ii) Ac-^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F (SEQ ID NO: 4), iii) ^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F-NH₂ (SEQ ID NO: 4), or iv) Ac-^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F-NH₂ (SEQ ID NO: 4).

In some embodiments, the ApoA-I mimetic polypeptide is formulated as a composition comprising a pharmaceutically acceptable carrier, excipient, or diluent or the formulation consists essentially of a pharmaceutically acceptable carrier, excipient, or diluent.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-B. ApoA1 mimetics 5A and 4F do not alter cellular proliferation of murine mammary cancer cells treated in vitro. (A) 4T1 or (B) E0771 murine mammary cancer cells.

FIG. 2: ApoA1 mimetics 5A and 4F modestly increase macrophage viability through time.

FIG. 3A-F: Treatment of macrophages with 5A, a peptide mimetic of ApoA1, results in increased anticancer functions. (A) 5A treated murine macrophages. (B) 5A increases capacity to infiltrate 4T1 tumor spheroids. (C) 5A treatment of murine macrophages. (D) 5A treatment of macrophages. (E) 5A treated macrophages co-cultured with activated T cells. (F) 5A treated macrophages co-cultured with T cells.

FIG. 4: T cells co-cultured with macrophages that had been pretreated with either 5A or 4F had increased capacity to kill E0771 mammary cancer cells.

FIG. 5: Treatment of human macrophages with 5A, a peptide mimetic of ApoA1, results in increased support of expanding CD8+ T cells

FIG. 6: Macrophages treated with 5A that are subsequently co-cultured with expanding T cells, results in increased expression of effector enzymes in T cells.

FIG. 7: Effects of 5A on macrophage support of T cell expansion require the presence of ABCA1.

FIG. 8: ApoA1 mimetics 5A and 4F work to increase dendritic cell support of T cell expansion.

FIG. 9: Treatment of mice bearing 4T1 mammary cancer metastases with 5A reduces metastatic burden.

### DETAILED DESCRIPTION OF THE INVENTION

Breast and other solid tumors respond poorly to immune therapy. Myeloid cells (MCs) such as macrophages contribute to resistance. Established clinical evidence links cholesterol to cancer outcomes, with MC function being regulated by cholesterol metabolism. We screened MC-expressed regulators of cholesterol homeostasis linked to survival and identified the cholesterol efflux protein ABCA1. ABCA1 activity increases anti-cancer functions of macrophages: enhancing tumor infiltration, decreasing angiogenic potential, reducing efferocytosis, and improving support of CD8+ T cell activity. Mechanistically, different AKT isoforms are involved, through both PI3K-dependent and independent mechanisms. Highlighting the clinical relevance of our findings are correlations between ABCA1 in macrophages and angiogenic potential, VEGFA, and CD8 T cell abundance and activity. The culmination of these activities was demonstrated through increased tumor growth and metastasis in mice lacking MC-expressed ABCA1. Tumors grown in these mice were also more resistant to immune therapy. Therefore, modulating ABCA1 activity within MCs may represent a novel approach to immune therapy.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, +10%, +20%, or +25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

The term “amino acid” refers to natural amino acids, unnatural amino acids, and amino acid analogs. Unless otherwise indicated, the term “amino acid” includes both D and L stereoisomers if the respective structure allows such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids, or non-natural amino acid include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), Nalkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“Octan”), ornithine (“Orn”), pentylglycine (“pG” or “PGly”), pipecolic acid, thioproline (“ThioP” or “tPro”), homoLysine (“hLys”), and homoArginine (“hArg”).

The terms “peptide mimetic” or “peptidomimetic” refer to a peptide-like molecule that emulates a sequence derived from a protein or peptide. A peptide mimetic or peptidomimetic can contain amino acids and/or non-amino acid components. Examples of peptidomimetics include chemically modified peptides, peptoids (side groups are appended to the nitrogen atom of the peptide backbone, rather than to the α-carbons), β-peptides (amino group bonded to the β carbon rather than the α-carbon), etc. Chemical modification includes one or more modifications at amino acid side groups, α-carbon atoms, terminal amine group, or terminal carboxy group. A chemical modification can be adding chemical moieties, creating new bonds, or removing chemical moieties. Modifications at amino acid side groups include, without limitation, acylation of lysine F-amino groups, N-alkylation of arginine, histidine, or lysine, alkylation of glutamic or aspartic carboxylic acid groups, lactam formation via cyclization of lysine F-amino groups with glutamic or aspartic acid side group carboxyl groups, hydrocarbon “stapling” (e.g., to stabilize alpha-helix conformations), and deamidation of glutamine or asparagine. Modifications of the terminal amine group include, without limitation, the desamino, N-lower alkyl, N-di-lower alkyl, constrained alkyls (e.g. branched, cyclic, fused, adamantyl) and N-acyl modifications. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, can be protected by protective groups known to the ordinarily skilled peptide chemist. The α-carbon of an amino acid can be mono- or dimethylated.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues can be divided into classes based on common side group properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class. An amino acid that has the D-configuration is denoted symbolically with a superscript capital “D” preceding its name or its standard abbreviation. For example, the D-configuration of alanine with be abbreviated as ^{D}alanine ^{D}Ala or ^{D}A; and it may also be indicated as D-alanine or a lower case single letter abbreviation, e.g., “a” for D-alanine.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, embodiment of the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. An appropriate “effective” amount in any individual case may be determined using techniques, such as a dose escalation study.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

As used herein, the term “subject” broadly refers to any animal, including but not limited to, human and non-human animals (e.g., dogs, cats, cows, horses, sheep, pigs, poultry, fish, crustaceans, etc.). As used herein, the term “patient” typically refers to a subject that is being treated for a disease or condition.

As used herein, the terms “administration” and “administering” refer to the act of giving a drug, prodrug, or other agent, or therapeutic treatment (e.g., synthetic peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the terms “peptide,” “polypeptide,” and “protein” are used interchangeably, and refer to a compound comprising amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “expression” as used herein is defined as the transcription and/or translation of a particular nucleotide sequence driven by its promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “transfected” or “transformed” or “infected” as used herein refers to a process by which exogenous nucleic acid is transferred or introduced into the host cell. A “transfected” or “transformed” or “infected” cell is one which has been transfected, transformed or transduced with exogenous nucleic acid. The cell includes the primary subject cell and its progeny.

Aspects of the invention are described, for example, in Bendre et al., Sci. Adv. 12,e adx5490 (2026). DOI:10.1126/sciadv.adx5490, including all supplemental materials, U.S. Patent Publication No. 2024/0309065 to Remaley et al.; U.S. Pat. No. 11,827,690 to Remaley et al.; Wolska et al., Cells 2021, 10 (3), 597, incorporated herein by reference in their entireties.

In some embodiments, the disclosure provides for methods of cell therapy using myeloid cell engineered to overexpress the ABCA1 protein. Generally, myeloid cells may be taken from a subject, caused to overexpress ABCA1, and reintroduced to the subject such that the activated engineered myeloid cells have greater efficacy in treating a cancer than a non-engineered myeloid cell. However, the myeloid cells may be derived from a source other than the subject in need thereof.

In some embodiments, a method of cell therapy for a subject having or is suspected of having a cancer comprises the steps of i) removing a population of myeloid cells from a subject having or suspected of having cancer, ii) transforming the population of myeloid cells with a polynucleotide encoding an ABCA1 gene to form a population of engineered myeloid cells, iii) expanding the population of engineered myeloid cells, and administering an effective amount of the expanding the population of engineered myeloid cells to the subject, thereby treating the cancer when present.

In some embodiments, a method of cell therapy for a subject having or suspected of having a cancer comprises the steps of i) providing a population of myeloid cells from a donor subject that does not have cancer, ii) transforming the population of myeloid cells with a polynucleotide encoding an ABCA1 gene to form a population of engineered myeloid cells, iii) expanding the population of engineered myeloid cells, and administering an effective amount of the expanded population of engineered myeloid cells to the subject, thereby treating the cancer when present. Preferably, the donor subject does not have cancer or otherwise does not have any known disease or condition (i.e., is a healthy donor subject).

In some embodiments, a method of cell therapy for a subject having or is suspected of having a cancer comprises the steps of i) removing a population of myeloid cells from a subject having or suspected of having cancer, ii) transforming the population of myeloid cells with a polynucleotide encoding an ABCA1 gene to form a population of engineered myeloid cells, iii) expanding the population of engineered myeloid cells, and administering an effective amount of the expanding the population of engineered myeloid cells to the subject, thereby treating the cancer when present, wherein the polynucleotide comprises nucleotide sequence that is at least about 75% identical to at least about 99% identical, or identical to SEQ ID NO: 5. For step i), the population of myeloid cells may be derived from a third party that does not have cancer or otherwise does not have any known disease or condition (i.e., is a health donor subject).

In some embodiments, a method of cell therapy for a subject having or is suspected of having a cancer comprises the steps of i) removing a population of myeloid cells from a subject having or suspected of having cancer, ii) transforming the population of myeloid cells with a polynucleotide encoding an ABCA1 gene to form a population of engineered myeloid cells, iii) expanding the population of engineered myeloid cells, and administering an effective amount of the expanding the population of engineered myeloid cells to the subject, thereby treating the cancer when present, wherein the polynucleotide comprises nucleotide sequence that is at least 95% identical to SEQ ID NO: 5. For step i), the population of myeloid cells may be derived from a third party that does not have cancer or otherwise does not have any known disease or condition (i.e., is a healthy donor subject).

In some embodiments, a method of cell therapy for a subject having or is suspected of having a cancer comprises the steps of i) removing a population of myeloid cells from a subject having or suspected of having cancer, ii) transforming the population of myeloid cells with a polynucleotide encoding an ABCA1 gene to form a population of engineered myeloid cells, iii) expanding the population of engineered myeloid cells, and administering an effective amount of the expanding the population of engineered myeloid cells to the subject, thereby treating the cancer when present, wherein the polynucleotide comprises nucleotide sequence that is at least 90% identical to SEQ ID NO: 5. For step i), the population of myeloid cells may be derived from a third party that does not have cancer or otherwise does not have any known disease or condition (i.e., is a healthy donor subject).

In some embodiments, a method of cell therapy for a subject having or is suspected of having a cancer comprises the steps of i) removing a population of myeloid cells from a subject having or suspected of having cancer, ii) transforming the population of myeloid cells with a polynucleotide encoding an ABCA1 gene to form a population of engineered myeloid cells, iii) expanding the population of engineered myeloid cells, and administering an effective amount of the expanding the population of engineered myeloid cells to the subject, thereby treating the cancer when present, wherein the polynucleotide comprises nucleotide sequence that is at least 95% identical to SEQ ID NO: 5. For step i), the population of myeloid cells may be derived from a third party that does not have cancer or otherwise does not have any known disease or condition (i.e., is a health donor subject).

In some embodiments, a method of expanding a population of genetically engineered myeloid cells in a subject diagnosed with cancer comprises the steps of administering to the subject an effective amount of a myeloid cell genetically engineered to overexpress an ABCA1 protein compared to a non-engineered myeloid cell, wherein the administered genetically engineered myeloid cell produces a population of progeny myeloid cells in the subject, and wherein the myeloid cell is from a subject having cancer.

In some embodiments, a method of expanding a population of genetically engineered myeloid cells in a subject diagnosed with cancer comprises the steps of administering to the subject an effective amount of a myeloid cell genetically engineered to overexpress an ABCA1 protein compared to a non-engineered myeloid cell, wherein the administered genetically engineered myeloid cell produces a population of progeny myeloid cells in the subject, and wherein the myeloid cell is from a subject that does not have cancer.

In some embodiments, a method of treating a subject having a disease, disorder or condition associated with an elevated expression of a tumor antigen, the method comprising administering to the subject an effective amount of a myeloid cell genetically modified to overexpress an ABCA1 protein compared to a non-engineered myeloid cell, thereby treating the subject, wherein the myeloid cell is from the subject having the disease, the disorder or the condition associated with an elevated expression of a tumor antigen.

In some embodiments, a method of treating a subject having a disease, disorder or condition associated with an elevated expression of a tumor antigen, the method comprising administering to the subject an effective amount of a myeloid cell genetically modified to overexpress an ABCA1 protein compared to a non-engineered myeloid cell, thereby treating the subject, wherein the myeloid cell is from a subject that does not have the disease, the disorder or the condition associated with an elevated expression of a tumor antigen.

Preferably, the polynucleotide comprises a nucleotide sequence encoding the ABCA1 gene is about 75% identical to about 99% identical to SEQ ID NO:5, or about 75% identical, about 80% identical, about 85% identical, about 90% identical, about 95% identical, about 96% identical, about 97% identical, about 98% identical, about 99% identical, or identical to SEQ ID NO: 5.

(SEQ ID NO: 5)
atggcttgttggcctcagctgaggttgctgctgtggaagaacctcactttcagaagaagacaaacatgtcagctgctgctggaagtggc
ctggcctctatttatcttcctgatcctgatctctgttcggctgagctacccaccctatgaacaacatgaatgccattttccaaataaagccat
gccctctgcaggaacacttccttgggttcaggggattatctgtaatgccaacaacccctgtttccgttacccgactcctggggaggctcc
cggagttgttggaaactttaacaaatccattgtggctcgcctgttctcagatgctcggaggcttcttttatacagccagaaagacaccagc
atgaaggacatgcgcaaagttctgagaacattacagcagatcaagaaatccagctcaaacttgaagcttcaagatttcctggtggacaa
tgaaaccttctctgggttcctgtatcacaacctctctctcccaaagtctactgtggacaagatgctgagggctgatgtcattctccacaagg
tatttttgcaaggctaccagttacatttgacaagtctgtgcaatggatcaaaatcagaagagatgattcaacttggtgaccaagaagtttct
gagctttgtggcctaccaagggagaaactggctgcagcagagcgagtacttcgttccaacatggacatcctgaagccaatcctgagaa
cactaaactctacatctcccttcccgagcaaggagctggctgaagccacaaaaacattgctgcatagtcttgggactctggcccagga
gctgttcagcatgagaagctggagtgacatgcgacaggaggtgatgtttctgaccaatgtgaacagctccagctcctccacccaaatct
accaggctgtgtctcgtattgtctgcgggcatcccgagggaggggggctgaagatcaagtctctcaactggtatgaggacaacaacta
caaagccctctttggaggcaatggcactgaggaagatgctgaaaccttctatgacaactctacaactccttactgcaatgatttgatgaa
gaatttggagtctagtcctctttcccgcattatctggaaagctctgaagccgctgctcgttgggaagatcctgtatacacctgacactcca
gccacaaggcaggtcatggctgaggtgaacaagaccttccaggaactggctgtgttccatgatctggaaggcatgtgggaggaactc
agccccaagatctggaccttcatggagaacagccaagaaatggaccttgtccggatgctgttggacagcagggacaatgaccactttt
gggaacagcagttggatggcttagattggacagcccaagacatcgtggcgtttttggccaagcacccagaggatgtccagtccagtaa
tggttctgtgtacacctggagagaagctttcaacgagactaaccaggcaatccggaccatatctcgcttcatggagtgtgtcaacctgaa
caagctagaacccatagcaacagaagtctggctcatcaacaagtccatggagctgctggatgagaggaagttctgggctggtattgtg
ttcactggaattactccaggcagcattgagctgccccatcatgtcaagtacaagatccgaatggacattgacaatgtggagaggacaaa
taaaatcaaggatgggtactgggaccctggtcctcgagctgacccctttgaggacatgcggtacgtctgggggggcttcgcctacttg
caggatgtggtggagcaggcaatcatcagggtgctgacgggcaccgagaagaaaactggtgtctatatgcaacagatgccctatccc
tgttacgttgatgacatctttctgcgggtgatgagccggtcaatgcccctcttcatgacgctggcctggatttactcagtggctgtgatcat
caagggcatcgtgtatgagaaggaggcacggctgaaagagaccatgcggatcatgggcctggacaacagcatcctctggtttagctg
gttcattagtagcctcattcctcttcttgtgagcgctggcctgctagtggtcatcctgaagttaggaaacctgctgccctacagtgatccca
gcgtggtgtttgtcttcctgtccgtgtttgctgtggtgacaatcctgcagtgcttcctgattagcacactcttctccagagccaacctggca
gcagcctgtgggggcatcatctacttcacgctgtacctgccctacgtcctgtgtgtggcatggcaggactacgtgggcttcacactcaa
gatcttcgctagcctgctgtctcctgtggcttttgggtttggctgtgagtactttgccctttttgaggagcagggcattggagtgcagtggg
acaacctgtttgagagtcctgtggaggaagatggcttcaatctcaccacttcggtctccatgatgctgtttgacaccttcctctatggggtg
atgacctggtacattgaggctgtctttccaggccagtacggaattcccaggccctggtattttccttgcaccaagtcctactggtttggcg
aggaaagtgatgagaagagccaccctggttccaaccagaagagaatatcagaaatctgcatggaggaggaacccacccacttgaag
ctgggcgtgtccattcagaacctggtaaaagtctaccgagatgggatgaaggtggctgtcgatggcctggcactgaatttttatgaggg
ccagatcacctccttcctgggccacaatggagcggggaagacgaccaccatgtcaatcctgaccgggttgttccccccgacctcggg
caccgcctacatcctgggaaaagacattcgctctgagatgagcaccatccggcagaacctgggggtctgtccccagcataacgtgct
gtttgacatgctgactgtcgaagaacacatctggttctatgcccgcttgaaagggctctctgagaagcacgtgaaggcggagatggag
cagatggccctggatgttggtttgccatcaagcaagctgaaaagcaaaacaagccagctgtcaggtggaatgcagagaaagctatct
gtggccttggcctttgtcgggggatctaaggttgtcattctggatgaacccacagctggtgtggacccttactcccgcaggggaatatg
ggagctgctgctgaaataccgacaaggccgcaccattattctctctacacaccacatggatgaagcggacgtcctgggggacaggatt
gccatcatctcccatgggaagctgtgctgtgtgggctcctccctgtttctgaagaaccagctgggaacaggctactacctgaccttggtc
aagaaagatgtggaatcctccctcagttcctgcagaaacagtagtagcactgtgtcatacctgaaaaaggaggacagtgtttctcagag
cagttctgatgctggcctgggcagcgaccatgagagtgacacgctgaccatcgatgtctctgctatctccaacctcatcaggaagcatg
tgtctgaagcccggctggtggaagacatagggcatgagctgacctatgtgctgccatatgaagctgctaaggagggagcctttgtgga
actctttcatgagattgatgaccggctctcagacctgggcatttctagttatggcatctcagagacgaccctggaagaaatattcctcaag
gtggccgaagagagtggggtggatgctgagacctcagatggtaccttgccagcaagacgaaacaggcgggccttcggggacaagc
agagctgtcttcgcccgttcactgaagatgatgctgctgatccaaatgattctgacatagacccagaatccagagagacagacttgctca
gtgggatggatggcaaagggtcctaccaggtgaaaggctggaaacttacacagcaacagtttgtggcccttttgtggaagagactgct
aattgccagacggagtcggaaaggattttttgctcagattgtcttgccagctgtgtttgtctgcattgcccttgtgttcagcctgatcgtgcc
accctttggcaagtaccccagcctggaacttcagccctggatgtacaacgaacagtacacatttgtcagcaatgatgctcctgaggaca
cgggaaccctggaactcttaaacgccctcaccaaagaccctggcttcgggacccgctgtatggaaggaaacccaatcccagacacg
ccctgccaggcaggggaggaagagtggaccactgccccagttccccagaccatcatggacctcttccagaatgggaactggacaat
gcagaacccttcacctgcatgccagtgtagcagcgacaaaatcaagaagatgctgcctgtgtgtcccccaggggcaggggggctgc
ctcctccacaaagaaaacaaaacactgcagatatccttcaggacctgacaggaagaaacatttcggattatctggtgaagacgtatgtg
cagatcatagccaaaagcttaaagaacaagatctgggtgaatgagtttaggtatggcggcttttccctgggtgtcagtaatactcaagca
cttcctccgagtcaagaagttaatgatgccatcaaacaaatgaagaaacacctaaagctggccaaggacagttctgcagatcgatttctc
aacagcttgggaagatttatgacaggactggacaccaaaaataatgtcaaggtgtggttcaataacaagggctggcatgcaatcagct
ctttcctgaatgtcatcaacaatgccattctccgggccaacctgcaaaagggagagaaccctagccattatggaattactgctttcaatca
tcccctgaatctcaccaagcagcagctctcagaggtggctctgatgaccacatcagtggatgtccttgtgtccatctgtgtcatctttgca
atgtccttcgtcccagccagctttgtcgtattcctgatccaggagcgggtcagcaaagcaaaacacctgcagttcatcagtggagtgaa
gcctgtcatctactggctctctaattttgtctgggatatgtgcaattacgttgtccctgccacactggtcattatcatcttcatctgcttcca
gcagaagtcctatgtgtcctccaccaatctgcctgtgctagcccttctacttttgctgtatgggtggtcaatcacacctctcatgtacccagc
ctcctttgtgttcaagatccccagcacagcctatgtggtgctcaccagcgtgaacctcttcattggcattaatggcagcgtggccacctttgt
gctggagctgttcaccgacaataagctgaataatatcaatgatatcctgaagtccgtgttcttgatcttcccacatttttgcctgggacgag
ggctcatcgacatggtgaaaaaccaggcaatggctgatgccctggaaaggtttggggagaatcgctttgtgtcaccattatcttgggac
ttggtgggacgaaacctcttcgccatggccgtggaaggggtggtgttcttcctcattactgttctgatccagtacagattcttcatcaggcc
cagacctgtaaatgcaaagctatctcctctgaatgatgaagatgaagatgtgaggcgggaaagacagagaattcttgatggtggaggc
cagaatgacatcttagaaatcaaggagttgacgaagatatatagaaggaagcggaagcctgctgttgacaggatttgcgtgggcattc
ctcctggtgagtgctttgggctcctgggagttaatggggctggaaaatcatcaactttcaagatgttaacaggagataccactgttacca
gaggagatgctttccttaacaaaaatagtatcttatcaaacatccatgaagtacatcagaacatgggctactgccctcagtttgatgccatc
acagagctgttgactgggagagaacacgtggagttctttgcccttttgagaggagtcccagagaaagaagttggcaaggttggtgagt
gggcgattcggaaactgggcctcgtgaagtatggagaaaaatatgctggtaactatagtggaggcaacaaacgcaagctctctacag
ccatggctttgatcggcgggcctcctgtggtgtttctggatgaacccaccacaggcatggatcccaaagcccggcggttcttgtggaat
tgtgccctaagtgttgtcaaggaggggagatcagtagtgcttacatctcatagtatggaagagtgtgaagctctttgcactaggatggca
atcatggtcaatggaaggttcaggtgccttggcagtgtccagcatctaaaaaataggtttggagatggttatacaatagttgtacgaatag
cagggtccaacccggacctgaagcctgtccaggatttctttggacttgcatttcctggaagtgttctaaaagagaaacaccggaacatg
ctacaataccagcttccatcttcattatcttctctggccaggatattcagcatcctctcccagagcaaaaagcgactccacatagaagact
actctgtttctcagacaacacttgaccaagtatttgtgaactttgccaaggaccaaagtgatgatgaccacttaaaagacctctcattacac
aaaaaccagacagtagtggacgttgcagttctcacatcttttctacaggatgagaaagtgaaagaaagctatgtatga.

In some embodiments, the polynucleotide encodes an ABCA1 protein that is about 75% identical to about 99% identical to SEQ ID NO:6, or about 75% identical, about 80% identical, about 85% identical, about 90% identical, about 95% identical, about 96% identical, about 97% identical, about 98% identical, about 99% identical, or identical to SEQ ID NO: 6.

(SEQ ID NO: 6)
MACWPQLRLLLWKNLTFRRRQTCQLLLEVAWPLFIFLILISVRLSYPPYEQHECHFPN
KAMPSAGTLPWVQGIICNANNPCFRYPTPGEAPGVVGNFNKSIVARLFSDARRLLLY
SQKDTSMKDMRKVLRTLQQIKKSSSNLKLQDFLVDNETFSGFLYHNLSLPKSTVDK
MLRADVILHKVFLQGYQLHLTSLCNGSKSEEMIQLGDQEVSELCGLPREKLAAAERV
LRSNMDILKPILRTLNSTSPFPSKELAEATKTLLHSLGTLAQELFSMRSWSDMRQEVM
FLTNVNSSSSSTQIYQAVSRIVCGHPEGGGLKIKSLNWYEDNNYKALFGGNGTEEDA
ETFYDNSTTPYCNDLMKNLESSPLSRIIWKALKPLLVGKILYTPDTPATRQVMAEVN
KTFQELAVFHDLEGMWEELSPKIWTFMENSQEMDLVRMLLDSRDNDHFWEQQLDG
LDWTAQDIVAFLAKHPEDVQSSNGSVYTWREAFNETNQAIRTISRFMECVNLNKLEP
IATEVWLINKSMELLDERKFWAGIVFTGITPGSIELPHHVKYKIRMDIDNVERTNKIK
DGYWDPGPRADPFEDMRYVWGGFAYLQDVVEQAIIRVLTGTEKKTGVYMQQMPY
PCYVDDIFLRVMSRSMPLFMTLAWIYSVAVIIKGIVYEKEARLKETMRIMGLDNSILW
FSWFISSLIPLLVSAGLLVVILKLGNLLPYSDPSVVFVFLSVFAVVTILQCFLISTLFSRA
NLAAACGGIIYFTLYLPYVLCVAWQDYVGFTLKIFASLLSPVAFGFGCEYFALFEEQG
IGVQWDNLFESPVEEDGFNLTTSVSMMLFDTFLYGVMTWYIEAVFPGQYGIPRPWYF
PCTKSYWFGEESDEKSHPGSNQKRISEICMEEEPTHLKLGVSIQNLVKVYRDGMKVA
VDGLALNFYEGQITSFLGHNGAGKTTTMSILTGLFPPTSGTAYILGKDIRSEMSTIRQN
LGVCPQHNVLFDMLTVEEHIWFYARLKGLSEKHVKAEMEQMALDVGLPSSKLKSK
TSQLSGGMQRKLSVALAFVGGSKVVILDEPTAGVDPYSRRGIWELLLKYRQGRTIILS
THHMDEADVLGDRIAIISHGKLCCVGSSLFLKNQLGTGYYLTLVKKDVESSLSSCRN
SSSTVSYLKKEDSVSQSSSDAGLGSDHESDTLTIDVSAISNLIRKHVSEARLVEDIGHE
LTYVLPYEAAKEGAFVELFHEIDDRLSDLGISSYGISETTLEEIFLKVAEESGVDAETS
DGTLPARRNRRAFGDKQSCLRPFTEDDAADPNDSDIDPESRETDLLSGMDGKGSYQV
KGWKLTQQQFVALLWKRLLIARRSRKGFFAQIVLPAVFVCIALVFSLIVPPFGKYPSL
ELQPWMYNEQYTFVSNDAPEDTGTLELLNALTKDPGFGTRCMEGNPIPDTPCQAGE
EEWTTAPVPQTIMDLFQNGNWTMQNPSPACQCSSDKIKKMLPVCPPGAGGLPPPQR
KQNTADILQDLTGRNISDYLVKTYVQIIAKSLKNKIWVNEFRYGGFSLGVSNTQALPP
SQEVNDAIKQMKKHLKLAKDSSADRFLNSLGRFMTGLDTKNNVKVWENNKGWHAI
SSFLNVINNAILRANLQKGENPSHYGITAFNHPLNLTKQQLSEVALMTTSVDVLVSIC
VIFAMSFVPASFVVFLIQERVSKAKHLQFISGVKPVIYWLSNFVWDMCNYVVPATLVI
IIFICFQQKSYVSSTNLPVLALLLLLYGWSITPLMYPASFVFKIPSTAYVVLTSVNLFIGI
NGSVATFVLELFTDNKLNNINDILKSVFLIFPHFCLGRGLIDMVKNQAMADALERFGE
NRFVSPLSWDLVGRNLFAMAVEGVVFFLITVLIQYRFFIRPRPVNAKLSPLNDEDEDV
RRERQRILDGGGQNDILEIKELTKIYRRKRKPAVDRICVGIPPGECFGLLGVNGAGKSS
TFKMLTGDTTVTRGDAFLNKNSILSNIHEVHQNMGYCPQFDAITELLTGREHVEFFA
LLRGVPEKEVGKVGEWAIRKLGLVKYGEKYAGNYSGGNKRKLSTAMALIGGPPVV
FLDEPTTGMDPKARRFLWNCALSVVKEGRSVVLTSHSMEECEALCTRMAIMVNGRF
RCLGSVQHLKNRFGDGYTIVVRIAGSNPDLKPVQDFFGLAFPGSVLKEKHRNMLQY
QLPSSLSSLARIFSILSQSKKRLHIEDYSVSQTTLDQVFVNFAKDQSDDDHLKDLSLHK
NQTVVDVAVLTSFLQDEKVKESYV.

For example, in some embodiments, a method of cell therapy for a subject having or is suspected of having a cancer comprises the steps of i) removing a population of myeloid cells from a subject having or suspected of having cancer, ii) transforming the population of myeloid cells with a polynucleotide encoding an ABCA1 protein to form a population of engineered myeloid cells, iii) expanding the population of engineered myeloid cells, and administering an effective amount of the expanding the population of engineered myeloid cells to the subject, thereby treating the cancer when present, wherein the ABCA1 protein is at least about 75% identical to at least about 99% identical, or identical to SEQ ID NO: 6.

In other embodiments, a method of cell therapy for a subject having or is suspected of having a cancer comprises the steps of i) providing a population of myeloid cells from a donor subject, ii) transforming the population of myeloid cells with a polynucleotide encoding an ABCA1 protein to form a population of engineered myeloid cells, iii) expanding the population of engineered myeloid cells, and administering an effective amount of the expanding population of engineered myeloid cells to the subject, thereby treating the cancer when present, wherein the ABCA1 protein is at least about 75% identical to at least about 99% identical, or identical to SEQ ID NO: 6, and the donor subject does not have cancer or otherwise does not have any known disease or condition (i.e., is a healthy donor subject).

In some embodiments, the myeloid cells comprise one or more of a hematopoietic stem cell, hemocytoblast, bone marrow cell, bone marrow stem cell, myeloid stem cell, myeloid progenitors, granulocyte, a monocyte, a macrophage, a dendritic cell, myeloid derived suppressor cell, monocytic myeloid derived suppressor cell, granulocytic myeloid derived suppressor cell, or a mast cell. In other embodiments, the myeloid cell is one or more of a megakaryocyte, an eosinophil, a basophil an erythrocyte, a monocyte and a neutrophil. It is known in the art that myeloid cells may be distinguished based on specific surface markers and proteins they express. For example, CD11B is a common marker of myeloid cells. Another example is that neutrophils may be characterized (using, for example, flow cytometry and immunohistochemistry) by CD66b, CD15, and CD16 expression, while monocytes by CD14 and CD16. Eosinophils may be identified by CD16, CD11b, and CRTH2, and basophils may be identified by CD123 and FcεRI expression. Dendritic cells, may be identified by markers like CD11c, CD86, and HLA-DR. Many myeloid cells are also commonly characterized by MHC Class II proteins, such as HLA-DR, HLA-DQ or HLA-DP.

Methods for expanding a cell population in vitro or ex vivo are known in the art and described, for example in U.S. Pat. No. 10,260,044 to Fong et al.; U.S. Pat. No. 12,060,577 to Wald et al.; U.S. Pat. No. 11,242,375 to Adusumilli et al.; U.S. Pat. No. 12,263,190 to Hasskarl et al. Further methos for expanding myeloid cells ex vivo are described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Publication No. 2006/0121005.

In some embodiments, the amount effective amount of the population of engineered myeloid cells administered to the subject comprises about 5×10⁵ cells to about 5×10⁸ cells, or about 5×10⁶ cells. In some embodiments, a dose of infused engineered myeloid cells comprises about 5×10⁵ cells to about 5×10⁸ cells, or about 5×10⁶ cells per kilogram of body weight. In some embodiments, the engineered myeloid cells are administered as a composition.

In some embodiments, a method of cell therapy for a subject having or is suspected of having a cancer comprises the steps of i) removing a population of myeloid cells from a subject having or suspected of having cancer, ii) transforming the population of myeloid cells with a polynucleotide encoding an ABCA1 protein to form a population of engineered myeloid cells, iii) expanding the population of engineered myeloid cells, and administering an effective amount of the expanding the population of engineered myeloid cells to the subject, thereby treating the cancer when present, wherein the ABCA1 protein is at least about 95% identical to SEQ ID NO: 6. Alternatively, for step i), the population of myeloid cells may be derived from a donor subject that does not have cancer or otherwise does not have any known disease or condition (i.e., is a healthy donor subject).

In some embodiments, a method of cell therapy for a subject having or is suspected of having a cancer comprises the steps of i) removing a population of myeloid cells from a subject having or suspected of having cancer, ii) transforming the population of myeloid cells with a polynucleotide encoding an ABCA1 protein to form a population of engineered myeloid cells, iii) expanding the population of engineered myeloid cells, and administering an effective amount of the expanding the population of engineered myeloid cells to the subject, thereby treating the cancer when present, wherein the ABCA1 protein comprises SEQ ID NO: 6. Alternatively, for step i), the population of myeloid cells may be derived from a donor subject that does not have cancer or otherwise does not have any known disease or condition (i.e., is a health donor subject).

The nucleic acid sequences coding for the desired molecules can be obtained using recombinant methods known in the art, such as, for example, by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of natural or synthetic nucleic acids encoding the ABCA1 gene is typically achieved by operably linking a nucleic acid encoding the ABCA1 protein or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence. See for example, U.S. Pat. No. 9,499,629 to June et al.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to CD11B promoter, CD11C promoter, LysM promoter, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence to which it is operatively linked when such expression is desired, or turning off the expression when expression is not desired. Examples of inducible promoters include, but are not limited to a metallothionine promoter, a glucocorticoid promoter, a progesterone promoter, a tetracycline promoter, a Gt(ROSA)26Sor promoter, and estrogen response element, or a promoter inducible by tamoxifen. Other suitable promoters are described in, for example, PCT Patent Publication No. 2025/090942 to Haldar et al; U.S. Pat. No. 9,340,798 to Trono et al.; U.S. Pat. No. 10,287,579 to Biffi et al.; U.S. Pat. No. 5,502,176 to Tenen et al.; U.S. Pat. No. 9,562,237 to Yang et al.; U.S. Pat. No. 7,709,625 to Li et al.; U.S. Pat. No. 7,198,909 to Ron et al.; U.S. Pat. No. 6,777,235 to Ong et al.; U.S. Patent Publication No. 2024/0209395 to Chang et al.; U.S. Patent Publication No. 20210277354 to Annoni et al.; and U.S. Patent Publication No. 2022/0378937 to Kohn et al.; Strathdee et al., PLoS One. 2006 Dec. 20; 1(1):e4. doi: 10.1371/journal.pone.0000004; Gong et al., Lung. 2019 Nov. 8; 197(6):819-823. doi: 10.1007/s00408-019-00286-5; Hara et al, Molecular Immunology, Volume 53, Issue 3, 2013, Pages 295-301, doi.org/10.1016/j.molimm.2012.08.004; and Dziennis et al., Blood. 1995 Jan. 15; 85(2):319-29.

In order to assess the expression of the desired polypeptide or portions thereof (i.e., ABCA1 protein), the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Patent Publication Nos. 2022/0001028 to Hatfield et al.; 2022/0125875 to Safieddine et al.; and 2020/0405883 to Bilic et al.; and also U.S. Pat. Nos. 5,350,674 and 5,585,362.

Chemical means for introducing a polynucleotide into a host cell include cationic or calcium phosphate (use of a phosphate containing buffer such as HEPES and a cationic salt such as strontium chloride, SrCl₂; calcium chloride, CaCl₂)), colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In some embodiments, the ABCA1 gene or portion thereof may be inserted into a target cell genome using clustered regularly interspaced short palindromic repeats (CRISPR) and CRISPR-associated (Cas) proteins as described, for example, in U.S. Pat. Nos. 10,266,850, 10,227,611, 10,000,772, and 10,113,167, and U.S. Patent Publication. No. 2019/0134227.

In general, the term “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR system may comprise, for example, CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains). In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

In some embodiments, a Cas nuclease and guide RNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr-mate sequence (optionally operably linked to the guide sequence), and a tracr sequence embedded within one or more intron sequences (e.g., each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of the CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to the cell.

In some embodiments, a vector comprises a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9.

In some embodiments the CRISPR enzyme is Cas9 and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce non-homologous end joining.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR complex to a target sequence may be assessed by any suitable assay. For example, the components of the CRISPR system sufficient to form the CRISPR complex, including the guide sequence to be tested, may be provided to the cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of the CRISPR complex, including the guide sequence to be tested and a control guide sequence different from the test guide sequence, and comparing binding or rate of cleavage at the target sequence between the test and control guide sequence reactions.

A guide sequence may be selected to target any target sequence. In some embodiments, the target sequence is a sequence within a genome of a cell. Exemplary target sequences include those that are unique in the target genome. In some embodiments, a guide sequence is selected to reduce the degree of secondary structure within the guide sequence. Secondary structure may be determined by any suitable polynucleotide folding algorithm.

In general, a tracr-mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences.

Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In some aspects, loop forming sequences for use in hairpin structures are four nucleotides in length and have the sequence GAAA. However, longer or shorter loop sequences may be used, as well as alternative sequences. In some embodiments, the sequences include a nucleotide triplet (for example, AAA), and an additional nucleotide (for example C or G). Examples of loop forming sequences include CAAA and AAAG. In some embodiments, the transcript or transcribed polynucleotide sequence has at least two or more hairpins. In some embodiments, the transcript has two, three, four or five hairpins. In a further embodiment, the transcript has at most five hairpins. In some embodiments, the single transcript further includes a transcription termination sequence, such as a polyT sequence, for example six T nucleotides.

The disclosure also provides for methods of treating certain diseases or conditions using one or more ApoA-I mimetic peptides.

In some embodiments, the disease or condition is a cancer. In some embodiments, the cancer treated is selected from the group consisting of: melanoma, kidney, hepatobiliary, head-neck squamous carcinoma, pancreatic, colon, bladder, glioblastoma, prostate, lung, and breast cancer. In some embodiments, the cancer is melanoma or breast cancer.

In some embodiments, the ApoA-I mimetic polypeptides comprise one or more of DWLKAFYDKVAEKLKEAFPDWAKAAYDKAAEKAKEAA (SEQ ID NO: 1), PVLDLFRELLNELLEALKQKLK (SEQ ID NO: 2), DWFKAFYDKVAEKFKEAF (SEQ ID NO: 3), and ^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F (SEQ ID NO: 4), wherein the superscript “D” indicates a D-amino acid, optionally, an acetyl moiety (Ac) is covalently bonded to an N-terminus nitrogen of the ApoA-I mimetic polypeptide, and optionally, an amine group is covalently bonded to a C-terminus carbon of the ApoA-I mimetic polypeptide. Other ApoA-I mimetic polypeptides are described in, for example, Reddy et al., Curr. Opin. Lipidol. 2014, 25, 304-308; and Gou et al., Br. J. Pharmacol. 2020, 177, 48.

In some embodiments, the ApoA-I mimetic polypeptides comprise one or more of Ac-DWLKAFYDKVAEKLKEAFPDWAKAAYDKAAEKAKEAA-NH₂ (SEQ ID NO: 1), PVLDLFRELLNELLEALKQKLK (SEQ ID NO: 2), Ac-DWFKAFYDKVAEKFKEAF-NH₂ (SEQ ID NO: 3), and Ac-^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F-NH₂ (SEQ ID NO: 4), wherein the superscript “D” indicates a D-amino acid, an acetyl moiety (Ac) is covalently bonded to an N-terminus nitrogen of the ApoA-I mimetic polypeptide, and an amine group is covalently bonded to a C-terminus carbon of the ApoA-I mimetic polypeptide.

In some embodiments, the ApoA-I mimetic polypeptide is about 50% identical to about 99% identical to any one of SEQ ID NOs:1-4.

In some embodiments, the ApoA-I mimetic polypeptide is about 50% identical, about 55% identical, about 60% identical, about 65% identical, about 70% identical, about 75% identical, about 80% identical, about 85% identical, about 90% identical, about 95% identical, and greater than about 95% identical to SEQ ID NO: 1.

In some embodiments, the ApoA-I mimetic polypeptide is about 50% identical, about 55% identical, about 60% identical, about 65% identical, about 70% identical, about 75% identical, about 80% identical, about 85% identical, about 90% identical, about 95% identical, and greater than about 95% identical to SEQ ID NO: 2.

In some embodiments, the ApoA-I mimetic polypeptide is about 50% identical, about 55% identical, about 60% identical, about 65% identical, about 70% identical, about 75% identical, about 80% identical, about 85% identical, about 90% identical, about 95% identical, and greater than about 95% identical to SEQ ID NO: 3.

In some embodiments, the ApoA-I mimetic polypeptide is about 50% identical, about 55% identical, about 60% identical, about 65% identical, about 70% identical, about 75% identical, about 80% identical, about 85% identical, about 90% identical, about 95% identical, and greater than about 95% identical to SEQ ID NO: 4.

In some embodiments, the ApoA-I mimetic polypeptide may include an acetyl moiety (Ac) covalently bonded to an N-terminus nitrogen and an amide group covalently bonded to C-terminus carbon of the ApoA-I mimetic polypeptide. For example, the ApoA-I mimetic polypeptide of SEQ ID NO: 3 can be i) DWFKAFYDKVAEKFKEAF (SEQ ID NO: 3) or ii) Ac-DWFKAFYDKVAEKFKEAF-NH₂ (SEQ ID NO: 3). The ApoA-I mimetic polypeptide of SEQ ID NO: 4 can be i) ^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F (SEQ ID NO: 4) or ii) Ac-^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F-NH₂ (SEQ ID NO: 4).

A person of ordinary skill in the art may determine at which amino acid positions a substitution may be made by techniques that are well known in the art, for example, by alanine scanning mutagenesis of the ApoA-I mimetic polypeptide and subsequent use of the resultant polypeptide in a functional assay to test its anti-cancer properties.

In some embodiments, the ApoA-I mimetic polypeptide is identical to any one of SEQ ID NOs:1-4.

In some embodiments, the N-terminus of the ApoA-I mimetic polypeptide is acylated. For example, the ApoA-I mimetic polypeptide may be Ac-DWLKAFYDKVAEKLKEAFPDWAKAAYDKAAEKAKEAA (SEQ ID NO: 1), Ac-PVLDLFRELLNELLEALKQKLK (SEQ ID NO: 4), Ac-DWFKAFYDKVAEKFKEAF (SEQ ID NO: 3), and Ac-^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F (SEQ ID NO: 4), wherein “Ac” is acetyl moiety is covalently bonded to an N-terminus nitrogen of the ApoA-I mimetic polypeptide and the superscript “D” indicates a D-amino acid.

In some embodiments, a method of treating cancer comprises administering an effective amount of an ApoA-I mimetic peptide to a subject having or suspected of having a cancer. wherein the ApoA-I mimetic peptide comprises one or more of:
DWLKAFYDKVAEKLKEAFPDWAKAAYDKAAEKAKEAA (SEQ ID NO: 1);
PVLDLFRELLNELLEALKQKLK (SEQ ID NO: 2);
DWFKAFYDKVAEKFKEAF (SEQ ID NO: 3); and
^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F (SEQ ID NO: 4), wherein a superscript “D” indicates a D-amino acid, optionally an acetyl moiety (Ac) is covalently bonded to an N-terminus nitrogen of the ApoA-I mimetic polypeptide, and optionally, an amine group is covalently bonded to a C-terminus carbon of the ApoA-I mimetic polypeptide.

In some embodiments, a method of treating cancer comprises administering an effective amount of an ApoA-I mimetic peptide to a subject having or suspected of having a cancer. wherein the ApoA-I mimetic peptide comprises one or more of:

(SEQ ID NO: 1)
Ac-DWLKAFYDKVAEKLKEAFPDWAKAAYDKAAEKAKEAA-NH₂;
(SEQ ID NO: 2)
Ac-PVLDLFRELLNELLEALKQKLK-N_{H2};
(SEQ ID NO: 3)
Ac-DWFKAFYDKVAEKFKEAF-NH₂;
and
(SEQ ID NO: 4)
AC-^{D}D^{D}W^{D}F^{D}K^{D}A^{D}F^{D}Y^{D}D^{D}K^{D}V^{D}A^{D}E^{D}K^{D}E^{D}K^{D}E^{D}A^{D}F-NH₂,

wherein a superscript “D” indicates a D-amino acid, an acetyl moiety (Ac) is covalently bonded to an N-terminus nitrogen of the ApoA-I mimetic polypeptide, and an amine group (NH₂) is covalently bonded to a C-terminus carbon of the ApoA-I mimetic polypeptide.

In other embodiments, a method of treating cancer comprises administering an effective amount of a composition comprising one or more ApoA-I mimetic peptides to a subject having or suspected of having a cancer, wherein the composition comprises one or more of a pharmaceutically acceptable carrier, excipient, or diluent, wherein the one or more ApoA-I mimetic peptides is:

(SEQ ID NO: 1)
Ac-DWLKAFYDKVAEKLKEAFPDWAKAAYDKAAEKAKEAA-NH₂;
(SEQ ID NO: 2)
Ac-PVLDLFRELLNELLEALKQKLK-N_{H2};
(SEQ ID NO: 3)
Ac-DWFKAFYDKVAEKFKEAF-NH₂;
and
(SEQ ID NO: 4)
AC-^{D}D^{D}W^{D}F^{D}K^{D}A^{D}F^{D}Y^{D}D^{D}K^{D}V^{D}A^{D}E^{D}K^{D}E^{D}K^{D}E^{D}A^{D}F-NH₂,

wherein a superscript “D” indicates a D-amino acid, an acetyl moiety (Ac) is covalently bonded to an N-terminus nitrogen of the ApoA-I mimetic polypeptide, and an amine group (NH₂) is covalently bonded to a C-terminus carbon of the ApoA-I mimetic polypeptide.

In other embodiments, a method of treating cancer comprises administering an effective amount of a composition comprising one or more ApoA-I mimetic peptides to a subject having or suspected of having a cancer, wherein the composition comprises one or more of a pharmaceutically acceptable carrier, excipient, or diluent, wherein the one or more ApoA-I mimetic peptides is:

(SEQ ID NO: 1)
Ac-DWLKAFYDKVAEKLKEAFPDWAKAAYDKAAEKAKEAA-NH₂;
(SEQ ID NO: 2)
Ac-PVLDLFRELLNELLEALKQKLK-N_{H2};
(SEQ ID NO: 3)
Ac-DWFKAFYDKVAEKFKEAF-NH₂;
and
(SEQ ID NO: 4)
AC-^{D}D^{D}W^{D}F^{D}K^{D}A^{D}F^{D}Y^{D}D^{D}K^{D}V^{D}A^{D}E^{D}K^{D}E^{D}K^{D}E^{D}A^{D}F-NH₂,

wherein a superscript “D” indicates a D-amino acid, and “Ac” is an acetyl moiety covalently bonded to an N-terminus nitrogen of the ApoA-I mimetic polypeptide, and NH₂ is an amine group that is covalently bonded to a C-terminus carbon of the ApoA-I mimetic polypeptide.

In some embodiments, the ApoA-I mimetic peptide or the therapeutic composition is administered in an amount, on a schedule, and for a duration sufficient to treat the patient. In some embodiments, the peptide is administered in an amount, on a schedule, and for a duration sufficient to decrease the size of one or more tumors by at least 5%, 10%, 15%, 20%, 25%, or more as compared to levels just prior to initiation of treatment. In other embodiments, the ApoA-I mimetic peptide is administered in an amount, on a schedule, and for a duration sufficient to decrease the severity of one or more symptoms caused by the cancer by at least 5%, 10%, 15%, 20%, 25%, or more as compared to levels just prior to initiation of treatment

In some embodiments, the ApoA-I mimetic peptide is administered in an amount, on a dosage schedule, and for a duration sufficient to decrease tumor size by at least 30%, 35%, 40%, 45%, 50%, or more. In particular embodiments, the ApoA-I mimetic polypeptide is administered in an amount, on a schedule, and for a time sufficient to reduce tumor size by at least 55%, 60%, 65%, 70%, or more.

The treatment can consist of a single dose or a plurality of doses over a period of time.

In certain embodiments, the ApoA-I mimetic peptide is produced recombinantly, for example using bacterial, yeast, or eukaryotic expression systems.

For recombinant production, a polynucleotide sequence encoding the ApoA-I mimetic peptide is inserted into an appropriate expression vehicle, that is, a vector which contains the necessary elements for the transcription and translation of the inserted coding sequence, or in the case of an RNA viral vector, the necessary elements for replication and translation. The expression vehicle is then transfected into a suitable target cell which will express the single or multi-domain peptide. Depending on the expression system used, the expressed peptide is then isolated by procedures well-established in the art. Methods for recombinant protein and peptide production are well known in the art.

To increase efficiency of production, the polynucleotide can be designed to encode multiple units of the single or multi-domain peptide separated by enzymatic cleavage sites. The resulting polypeptide can be cleaved (e.g., by treatment with the appropriate enzyme) in order to recover the peptide units. This can increase the yield of peptides driven by a single promoter. In some embodiments, a polycistronic polynucleotide can be designed so that a single mRNA is transcribed which encodes multiple peptides, each coding region operatively linked to a cap-independent translation control sequence, for example, an internal ribosome entry site (IRES). When used in appropriate viral expression systems, the translation of each peptide encoded by the mRNA is directed internally in the transcript, for example, by the IRES. Thus, the polycistronic construct directs the transcription of a single, large polycistronic mRNA which, in turn, directs the translation of multiple, individual peptides. This approach eliminates the production and enzymatic processing of polypeptides and can significantly increase yield of peptide driven by a single promoter.

As noted herein, a variety of host-expression vector systems can be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus (CaMV) or tobacco mosaic virus (TMV)) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter) and the like can be used. When cloning in insect cell systems, promoters such as the baculovirus polyhedron promoter can be used. When cloning in plant cell systems, promoters derived from the genome of plant cells (e.g., heat shock promoters, the promoter for the small subunit of RUBISCO, the promoter for the chlorophyll a/b binding protein) or from plant viruses (e.g., the 35S RNA promoter of CaMV, the coat protein promoter of TMV) can be used. When cloning in mammalian cell systems, promoters derived from the genome of mammalian cells (e.g., metallothionein promoter) or from mammalian viruses (e.g., the adenovirus late promoter, the vaccinia virus 7.5 K promoter) can be used.

In certain other embodiments, the ApoA-I mimetic peptide is produced by chemical synthesis. In some embodiments, the ApoA-I mimetic peptide is produced using liquid phase peptide synthesis techniques. In some other embodiments, the peptide is produced using solid phase peptide synthesis techniques.

ApoA-I mimetic peptides having either the D- or L-configuration can be synthesized by automated solid phase procedures well known in the art (such as solid-phase peptide synthesis or liquid-phase peptide synthesis). Suitable syntheses can be performed by utilizing “Boc” or “Fmoc” procedures. Techniques and procedures for solid phase synthesis are well-known in the art. The single and multi-domain peptides can also be prepared by way of segment condensation, as described, for example, in Liu et al., Tetrahedron Lett. 37:933-936, 1996; Baca et al., J. Am. Chem. Soc. 117: 1881-1887, 1995; Tam et al., Int. J. Peptide Protein Res. 45:209-216, 1995; Schnolzer and Kent, Science 256:221-225, 1992; Liu and Tam, J. Am. Chem. Soc. 116:4149-4153,1994; Liu and Tam, Proc. Natl. Acad. Sci. USA 91:6584-6588, 1994; and Yamashiro and Li, Int. J. Peptide Protein Res. 31:322-334, 1988). This is particularly the case with glycine containing peptides. Other methods useful for synthesizing the single and multi-domain peptides of the disclosure are described in Nakagawa et al., J. Am. Chem. Soc. 107:7087-7092, 1985.

Additional exemplary techniques known to those of ordinary skill in the art of peptide and peptide analog synthesis are taught by Bodanszky, M. and Bodanszky, A., The Practice of Peptide Synthesis, Springer Verlag, New York, 1994; and by Jones, J., Amino Acid and Peptide Synthesis, 2nd ed., Oxford University Press, 2002. The Bodanszky and Jones references detail the parameters and techniques for activating and coupling amino acids and amino acid derivatives. Moreover, the references teach how to select, use and remove various useful functional and protecting groups.

ApoA-I mimetic peptides having either the D- or L-configuration can also be purchased from commercial suppliers of synthetic peptides. Such suppliers include, for example, GenScript Biotech (Piscataway, NJ), ProteoGenix (Schiltigheim, France), Advanced ChemTech (Louisville, KY), Applied Biosystems (Foster City, CA), Bachem (Torrance, CA), Anaspec (San Jose, CA), and Cell Essentials (Boston, MA).

The ApoA-I mimetic peptides or peptide analogs of the disclosure can be purified by many techniques well known in the art, such as reverse phase chromatography, high performance liquid chromatography, ion exchange chromatography, size exclusion chromatography, affinity chromatography, gel electrophoresis, and the like. The actual conditions used to purify a particular single or multi-domain peptide or peptide analog will depend, in part, on synthesis strategy and on factors such as net charge, hydrophobicity, hydrophilicity, and the like, and will be apparent to those of ordinary skill in the art.

In various embodiments, the ApoA-I mimetic peptide may further comprise a purification tag. In some embodiments, the purification tag is a polyhistidine-tag, a myc-tag, or an HA-tag.

In some embodiments, a subject having cancer, a clinical procedure or cancer therapy can be administered to the subject along with the methods described herein. Exemplary therapies or procedures include but are not limited to surgery, radiation therapy, chemotherapy, hormone therapy, targeted therapy, and/or administration (sequentially or concurrently with the methods described herein) of one or more of a second active agent such as: Abitrexate (Methotrexate), Abraxane (Paclitaxel Albumin-stabilized Nanoparticle Formulation), Ado-Trastuzumab Emtansine, Afinitor (Everolimus), Anastrozole, Aredia (Pamidronate Disodium), Arimidex (Anastrozole), Aromasin (Exemestane), Camizestrant, Capecitabine, Clafen, (Cyclophosphamide), Cyclophosphamide, Cytoxan (Cyclophosphamide), Docetaxel, Doxorubicin Hydrochloride, elacestrant, Ellence (Epirubicin Hydrochloride), Epirubicin Hydrochloride, Eribulin Mesylate, Everolimus, Exemestane, 5-FU (Fluorouracil Injection), Fareston (Toremifene), Faslodex (Fulvestrant), Femara (Letrozole), Fluorouracil Injection, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ibrance (Palbociclib), Imlunestrant, Ixabepilone, Ixempra (Ixabepilone), Kadcyla (Ado-Trastuzumab Emtansine), Kisqali (Ribociclib), Lapatinib Ditosylate, Letrozole, Megestrol Acetate, Methotrexate, Methotrexate LPF (Methotrexate), Mexate (Methotrexate), Mexate-AQ (Methotrexate), Neosar (Cyclophosphamide), Neratinib Maleate, Nerlynx (Neratinib Maleate), Nolvadex (Tamoxifen Citrate), Paclitaxel, Paclitaxel Albumin-stabilized Nanoparticle Formulation, Palbociclib, Pamidronate Disodium, Perjeta (Pertuzumab), Pertuzumab, Ribociclib, Tamoxifen Citrate, Taxol (Paclitaxel), Taxotere (Docetaxel), Thiotepa, Toremifene, Trastuzumab, Tykerb (Lapatinib Ditosylate), Velban (Vinblastine Sulfate), Velsar (Vinblastine Sulfate), Vinblastine Sulfate, Xeloda (Capecitabine), and Zoladex (Goserelin Acetate). Also see, for example, U.S. Pat. No. 10,350,207 to Hergenrother et al., and U.S. Patent Publication No. 2022/0226311 to Hergenrother et al. In other embodiments, the second active agent may be one or more checkpoint inhibitors such as, but not limited to, atezolimumab, avelumab, bevacizumab, BMS986016, BMS986156, CP870893, durvalumab, FAZ053, GSK3174998, GWN323, IMP321, ipilimumab, JTX-2011, LAG525, MBG453, MEDI0562, MEDI0680, MEDI6469, MOXR0916, nivolumab, PDR001, pembrolizumab, PF-04518600, REGN2810, REGN3767, R07009789, tremelimumab, TSR022, urelumab, utomilumab, or a combination thereof. Also see U.S. Pat. No. 9,856,320 to Cogswell et al.; U.S. Pat. No. 10,660,954 to Mitchell et al; and U.S. Patent Publication No. 2016/0185870. Other second active agents include the use of CAR T-cells such as described in U.S. Pat. No. 7,402,431 to Har-Noy et al. and 9,499,629 to June et al. or other cell based therapies such as described in U.S. Pat. No. 12,281,132 to Rosen et al.; U.S. Pat. No. 12,473,533 to Marson et al.; and U.S. Pat. No. 10,273,452 to Chambers et al.

In some embodiments, method of treatment of a cancer may include a suitable substance able to target intracellular proteins, small molecules, or nucleic acid molecules alone or in combination with an appropriate carrier or vehicle, including, but not limited to, an antibody or functional fragment thereof, (e.g., Fab′, F(ab′)2, Fab, Fv, rlgG, and scFv fragments and genetically engineered or otherwise modified forms of immunoglobulins such as intrabodies and chimeric antibodies), small molecule inhibitors of the protein, chimeric proteins or peptides, gene therapy for inhibition of transcription, or an RNA interference (RNAi)-related molecule or morpholino molecule able to inhibit gene expression and/or translation. In one embodiment the inhibitor is an RNAi-related molecule such as an siRNA or an shRNA for inhibition of translation. An RNA interference (RNAi) molecule is a small nucleic acid molecule, such as a short interfering RNA (siRNA), a double-stranded RNA (dsRNA), a micro-RNA (miRNA), or a short hairpin RNA (shRNA) molecule, that complementarily binds to a portion of a target gene or mRNA so as to provide for decreased levels of expression of the target. In some embodiments, any of the preceding compounds may be administered to a subject concurrently or sequentially to the methods described herein.

In some embodiments, a method of treating cancer in a subject comprising administering to the subject a therapeutically effective amount of a population of activated myeloid cells or a composition thereof, wherein the population of activated myeloid cells treat the cancer, wherein the population of activated myeloid cells are formed by the steps of: i) contacting a population of myeloid cells in vitro one with one or more ApoA-I mimetic polypeptides to form the population of engineered myeloid cells; ii) expanding the population of activated myeloid cells; and iii) administering the effective amount of the expanded population of activated myeloid cells to the subject, thereby treating the cancer, wherein the one or more ApoA-I mimetic polypeptides comprise an amino acid sequence that is at least about 85% identical to: DWLKAFYDKVAEKLKEAFPDWAKAAYDKAAEKAKEAA (SEQ ID NO: 1); PVLDLFRELLNELLEALKQKLK (SEQ ID NO: 2); DWFKAFYDKVAEKFKEAF (SEQ ID NO: 3); or ^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F (SEQ ID NO: 4); wherein superscript “D” indicates a D-amino acid, and optionally, an acetyl moiety (Ac) is covalently bonded to an N-terminus nitrogen of the ApoA-I mimetic polypeptide, and optionally, an amine group is covalently bonded to a C-terminus of the ApoA-I mimetic polypeptide.

In some embodiments, a method of treating cancer in a subject comprising administering to the subject a therapeutically effective amount of a population of activated myeloid cells or a composition thereof, wherein the population of activated myeloid cells treat the cancer, wherein the population of activated myeloid cells are formed by the steps of: i) contacting a population of myeloid cells in vitro one with one or more ApoA-I mimetic polypeptides to form the population of engineered myeloid cells; ii) expanding the population of activated myeloid cells; and iii) administering the effective amount of the expanded population of activated myeloid cells to the subject, thereby treating the cancer, wherein the one or more ApoA-I mimetic polypeptides comprise an amino acid sequence that is at least about 85% identical to: Ac-DWLKAFYDKVAEKLKEAFPDWAKAAYDKAAEKAKEAA-NH₂ (SEQ ID NO: 1); Ac-PVLDLFRELLNELLEALKQKLK-NH₂ (SEQ ID NO: 2); Ac-DWFKAFYDKVAEKFKEAF-NH₂ (SEQ ID NO: 3); or Ac-^{D}D^{D}W^{D}F^{D}K^{D}A^{D}F^{D}Y^{D}D^{D}K^{D}V^{D}A^{D}E^{D}K^{D}F^{D}K^{D}E^{D}A^{D}F-NH₂ (SEQ ID NO: 4); wherein superscript “D” indicates a D-amino acid, and optionally, an acetyl moiety (Ac) is covalently bonded to an N-terminus nitrogen of the ApoA-I mimetic polypeptide, and optionally, an amine group is covalently bonded to a C-terminus of the ApoA-I mimetic polypeptide.

In some embodiments, a method of cell therapy comprises administering to the subject a therapeutically effective amount of a population of activated myeloid cells or a composition thereof, wherein the population of activated myeloid cells treat the cancer, wherein the population of activated myeloid cells are formed by the steps of: i) contacting a population of myeloid cells in vitro one with one or more ApoA-I mimetic polypeptides to form the population of engineered myeloid cells; ii) expanding the population of activated myeloid cells; and iii) administering the effective amount of the expanded population of activated myeloid cells to the subject, thereby treating the cancer, wherein the one or more ApoA-I mimetic polypeptides comprise an amino acid sequence that is at least about 85% identical to: DWLKAFYDKVAEKLKEAFPDWAKAAYDKAAEKAKEAA (SEQ ID NO: 1), wherein optionally, an acetyl moiety (Ac) is covalently bonded to an N-terminus nitrogen of the ApoA-I mimetic polypeptide, and optionally, an amine group is covalently bonded to a C-terminus of the ApoA-I mimetic polypeptide.

In some embodiments, a method of cell therapy comprises administering to the subject a therapeutically effective amount of a population of activated myeloid cells or a composition thereof, wherein the population of activated myeloid cells treat the cancer, wherein the population of activated myeloid cells are formed by the steps of: i) contacting a population of myeloid cells in vitro one with one or more ApoA-I mimetic polypeptides to form the population of engineered myeloid cells; ii) expanding the population of activated myeloid cells; and iii) administering the effective amount of the expanded population of activated myeloid cells to the subject, thereby treating the cancer, wherein the one or more ApoA-I mimetic polypeptides comprise the amino acid sequence according to DWLKAFYDKVAEKLKEAFPDWAKAAYDKAAEKAKEAA (SEQ ID NO: 1) wherein optionally, an acetyl moiety (Ac) is covalently bonded to an N-terminus nitrogen of the ApoA-I mimetic polypeptide, and optionally, an amine group is covalently bonded to a C-terminus of the ApoA-I mimetic polypeptide.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection, or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro-emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like, into the compositions. In addition, prolonged absorption of the injectable pharmaceutical form may be brought about by inclusion of agents that delay absorption such as aluminum monostearate and gelatin.

In some cases, in order to prolong the effect of a drug, it is desirable to slow the absorption of the drug from subcutaneous or intramuscular injection. This may be accomplished by use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13^{th} ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

A suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent, concurrently or sequentially.

In some embodiments, compositions are formulated for administration by any suitable route, including but not limited to, orally (e.g., such as in the form of tablets, capsules, granules or powders), sublingually, bucally, parenterally (such as by subcutaneous, intravenous, intramuscular, intradermal, or intrasternal injection or infusion (e.g., as sterile injectable aqueous or non-aqueous solutions or suspensions, etc.)), nasally (including administration to the nasal membranes, such as by inhalation spray), topically (such as in the form of a cream or ointment), transdermally (such as by transdermal patch), rectally (such as in the form of suppositories), or by surgical implantation at a particular site, etc.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

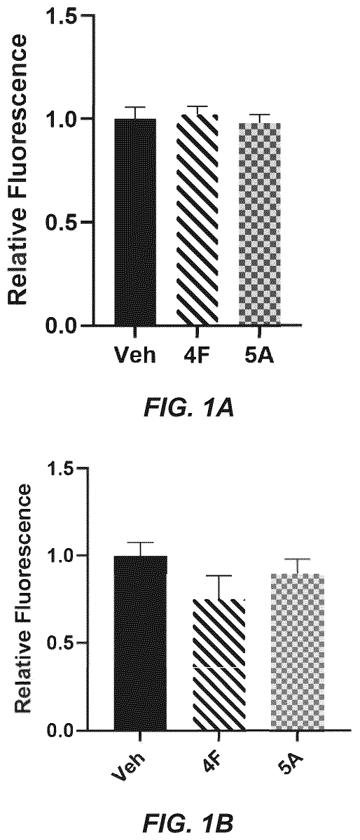

Example 1. ApoA1 mimetics 5A and 4F do not alter cellular proliferation of marine mammary cancer cells treated in vitro. As shown in FIG. 1, (A) 4T1 or (B) E0771 murine mammary cancer cells were cultured in vitro and treated with indicated ligands for 4 or 6 days respectively. Cellular proliferation was assessed by DNA quantity. No statistically significant differences were found.

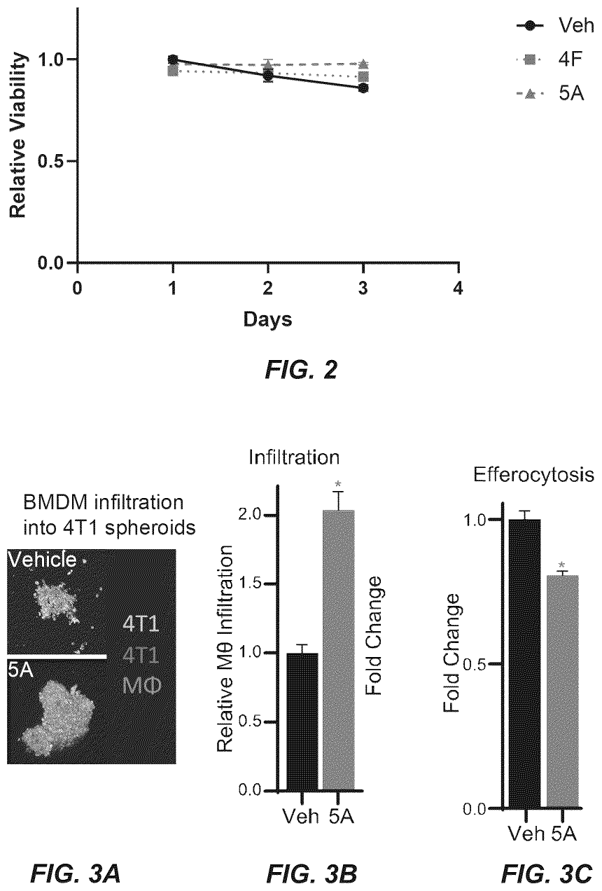

Example 2. ApoA1 mimetics 5A and 4F modestly increase macrophage viability through time. As shown in FIG. 2, Bone marrow derived macrophages were treated with indicated ligands and viability assessed using the resazurin assay. 2-way ANOVA identified an interaction (P=0.0271), but a multiple comparison posthoc test did not show any significant differences

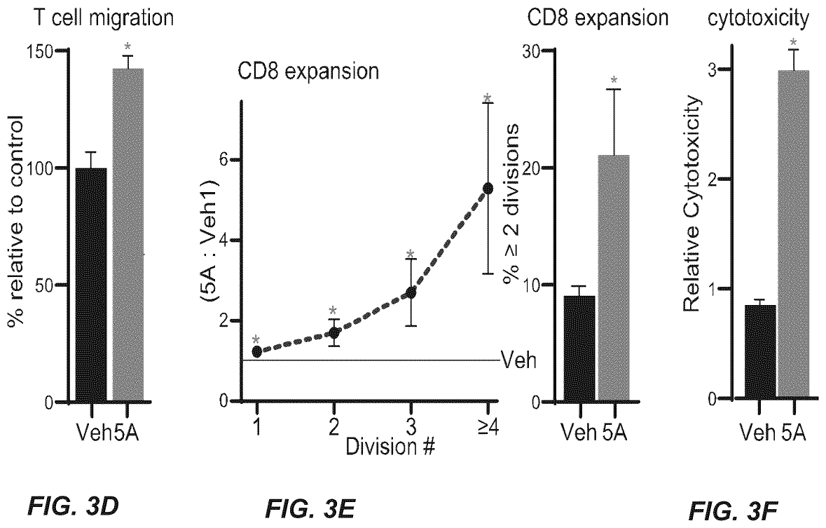

Example 3. Treatment of macrophages with 5A, a peptide mimetic of ApoA1, results in increased anticancer functions. As shown in FIG. 3, (A) 5A treated murine macrophages had significantly increased infiltration into 4T1 tumor spheroids compared to vehicle treated macrophages. Representative immunofluorescence images shown here. 4T1 cells were double stained yellow and blue, while macrophages were stained magenta. (B) 5A increases capacity to infiltrate 4T1 tumor spheroids. Macrophages were treated with vehicle (veh) or 5A for 24h and then overlayed onto 4T1 mammary cancer spheroids. Infiltration into spheroids was quantified using flow cytometry as described in Bendre et al, Science Advances, 2026. (C) 5A treatment of murine macrophages results in decreased efferocytosis, (D) 5A treatment of macrophages which are subsequently co-cultured with T cells results in increased T cell migration. (E) 5A treated macrophages that are subsequently co-cultured with activated T cells results in enhanced CD8+ T cell expansion. A ratio of 5A: veh for each division is indicated on the left, and a bar graph showing divisions of 2 or greater on the right. (F) 5A treated macrophages that are subsequently co-cultured with T cells results in enhanced CD8+ capacity to kill E0771 cancer cells expressing the OVA peptide.

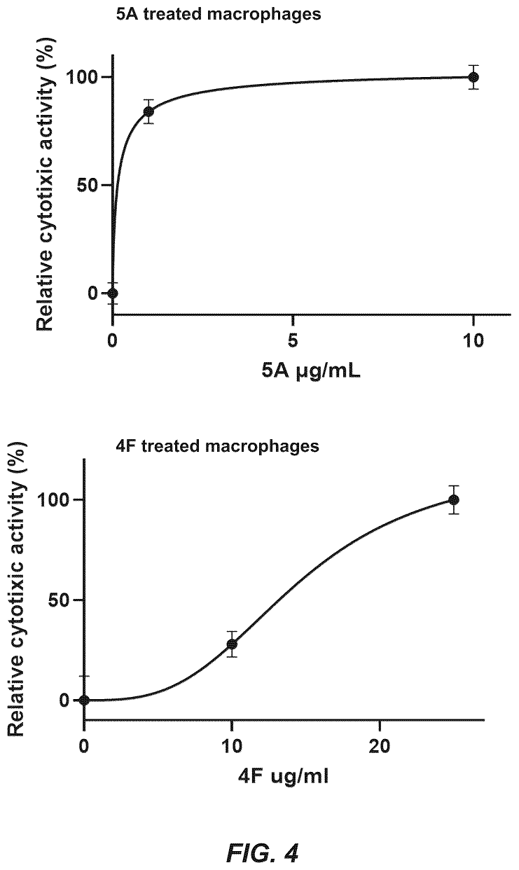

Example 4. T cells co-cultured with macrophages that had been pretreated with either 5A or 4F had increased capacity to kill E0771 mammary cancer cells. As shown in FIG. 4, anti-cancer cytotoxicity of T cells is influenced after coculture with BMDMs treated with either 5A or 4F. BMDMs were transfected or treated as indicated, treated with OVA and co-cultured with OT-I T cells. Equal numbers of T cells were then co-cultured with 1:1 E0771:E0771-OVA cells, each stained with a different color. Ratios between OVA− and OVA+E0771 cells were established and normalized between vehicle control and the highest mean. Data was fit to a 4-parameter variable slope regression model.

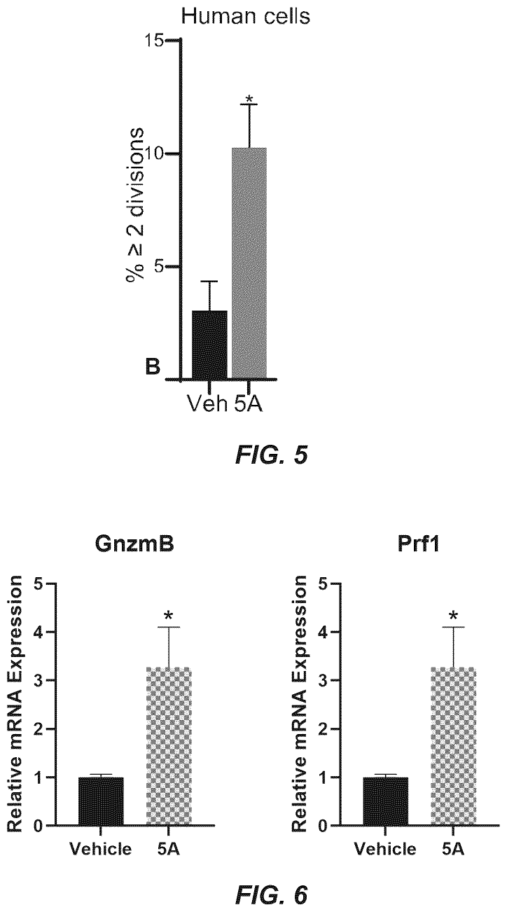

Example 5. Treatment of human macrophages with 5A, a peptide mimetic of ApoA1, results in increased support of expanding CD8+ T cells. As shown in FIG. 5, macrophages derived from human PBMCs were treated with 5A prior to co-culture with activated T cells from the same volunteer. T cells with equal to or greater than 1 division were quantified and displayed here. The asterisk denotes statistically significant difference (P>0.05).

Example 6. Macrophages treated with 5A that are subsequently co-cultured with expanding T cells, results in increased expression of effector enzymes in T cells. As shown in FIG. 6, macrophages were treated with 5A for 24 hrs before being washed and co-cultured with expanding T cells. T cells were then isolated and mRNA expression of granzyme B (GnzmB) and perforin 1 (Prf1) was assessed by quantitative PCR. Results are displayed as mRNA expression relative to vehicle control.

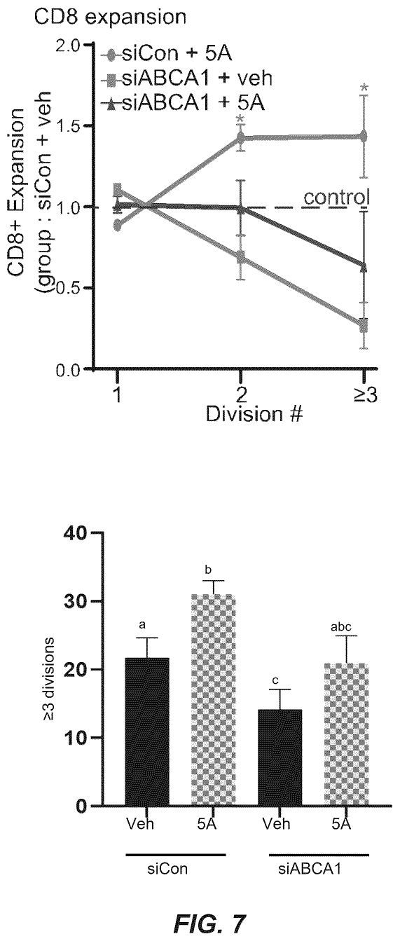

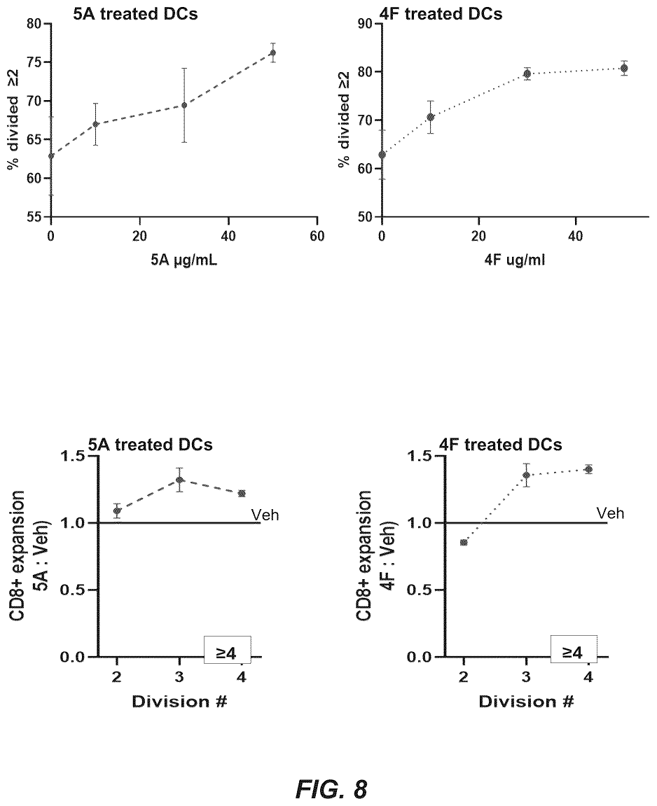

Example 7. Effects of 5A on macrophage support of T cell expansion require the presence of ABCA1. As shown in FIG. 7, siRNA against ABCA1 in macrophages attenuated the effects of 5A on CD8+ cell expansion. Macrophages were transfected with control (siCon) or siRNA against ABCA1 (siABCA1), prior to their treatment with either vehicle (veh) or 5A. Macrophages were then washed and co-cultured with activated T cells. T cell expansion was assessed by flow cytometry. Results on top are presented as a ratio to between experimental group and siCon+Veh (dashed line). Asterisks denote statistically significant differences. Results on bottom are presented as CD8+ T cells that had gone through more than 2 divisions (different letters denote statistically significant differences).

Example 8. ApoA1 mimetics 5A and 4F work to increase dendritic cell support of T cell expansion. As shown in FIG. 8, murine bone marrow derived dendritic cells (DCs) were treated with 5A or 4F as indicated, prior to incubation with activated T cells. Upper panels: CD8 T cell expansion indicated by percentage divided 2 or more times, as a reflection of dose. Bottom panels: Ratio of mimetic: Veh CD8+ divided cells at each generation (division number). Solid line represents vehicle (Veh) control. 5A and 4F were administered at 50 μg/mL for the bottom panels.

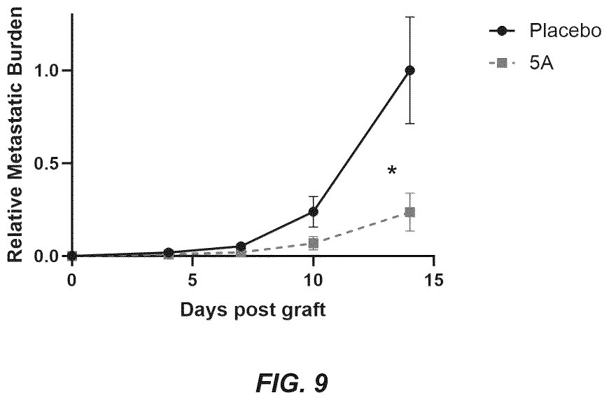

Example 9. Treatment of mice bearing 4T1 mammary cancer metastases with 5A reduces metastatic burden. As shown in FIG. 9, mice were grafted with 4T1 cells intravenously and metastatic lesions allowed to form. Twice a day treatment with 5A commenced on day 3 and metastatic burden was followed through time by bioluminescent imaging (IVIS). Two-way ANOVA followed by Šidák post-hoc (P<0.05 N=14/group)*s indicate statistical significance.

### Example 10: Materials and Methods

### Reagents

PSC833 was purchased from Sigma Aldrich (Cat No 121584-18-7) and dissolved in DMSO. OVA peptide (257-264, chicken) was purchased from Sigma Aldrich (Cat No S7951-1MG) and dissolved in PBS, anti-CD3 and anti-CD28 antibodies were purchased from BD Biosciences, Cat No 553057 and 553294 respectively. LPS was purchased from ThermoFisher (Cat No: 00-4973-03). Antibodies for flow cytometry were purchased from BD Biosciences and ThermoFisher. FACS Buffer is 2% Fetal Bovine Serum. CFSE was purchased from Biolegend, Cell Trace Red, and Cell Trace Violet from ThermoFisher. FBS was from Gibco Cytvia HyClone. Non-essential amino acids, Sodium Pyruvate, Penicillin/Streptomycin, PBS, cell stripper solution, RPMI-1640 and trypsin were from Corning. DMEM was from Fisher Sciences. siRNAs against ABCA1 (siRNA ID: SASI_Mm01_00075553, SASI_Mm01_00075555), AKT1(siRNA ID: SASI_Mm02_00316647), AKT2 (siRNA ID: SASI_Mm02_00304936) and AKT3(siRNA ID: SASI_Mm01_00200790) were purchased from Sigma Aldrich.

### Cell Culture

E0771 and 4T1-luc cells were a gift from Mark Dewhirst (Duke University) and cultured in DMEM F12 (ThermoFisher) supplemented with 10% FBS, 1% non-essential amino acids, 1% sodium pyruvate and 1% penicillin/streptomycin. E0771-OVA cells were a gift from Jun Yan (University of Louisville) and grown in the above-described conditions. All cell lines were tested for Mycoplasma contamination.

### Isolation of Bone Marrow-Derived Macrophages (BMDMs)

Bone marrow cells were collected from the mouse tibia and femur by flushing with complete DMEM. Cells were then passed through a 70 μm filter. Complete Macrophage Media consisting of DMEM supplemented with 25 ng/mL of M-CSF (PeproTech) was added on day 2, and media was replaced on day 6 and every 2-3 days after that. BMDMs were harvested between day 10-day 12 using cell stripper solution and were used for subsequent experiments.

### Animal Studies

All animal protocols were approved by the Institutional Animal Care and Use Committee (IACUC) at the University of Illinois Urbana Champaign (protocols 20032, 20236, 23021 and 23217). BALB/C and C57BL6/J mice were purchased from either Charles River Laboratories or The Jackson Laboratory. Abca1^{fl/fl} and LysMCre+ mice were purchased from The Jackson Laboratory, and bred at the University of Illinois to obtain Abca1^{fl/fl}; LysMCre⁺ and Abca1^{+/+};LysMCre⁺ mice. Mice were housed with ad libitum access to food and water and 12 h light and dark cycles.

Studies with Human Tissue:

Human tissue or blood samples were de-identified prior to procurement by researchers, and thus deemed not human subject research by the Institutional Review Board of the University of Illinois Urbana Champaign.

### In-Vivo Macrophage Infiltration Study

300,000 4T1 cells were grafted orthotopically in the mammary fat pad and were allowed to grow for 3 days. BMDMs harvested from syngeneic mice were transfected with either a control or siRNA against ABCA1 which were then injected in the subcutaneous space for consecutive 4 days. Tumors were then weighed post-euthanasia and analyzed via flow cytometry.

Abca1^{fl/fl};LysMCre⁺ E0771 Primary Tumor Study

We obtained Abca1^{fl/fl} mice from The Jackson Laboratory and bred them with LysMCre+ mice to generate a myeloid-specific ABCA1 knockout cell line. These mice were genotyped using the protocol provided by the Jackson Laboratory. 200,000 E0771 cells were grafted in the mammary fat pad of age-matched females 2-5 months old. Tumors were measured over time using a digital caliper. Tumors were then weighed post-euthanasia and analyzed via flow cytometry.

Abca1^{fl/fl};LysMCre⁺ E0771 Metastasis Study

150,000 E0771 cells were injected intravenously via the tail vein into age-matched females 2-5 months old. 2 weeks post graft, mice were euthanized. Lungs were analyzed using histology and flow cytometry.

Abca1^{fl/fl};LysMCre⁺ BI6-F10 Primary Tumor Study

200,000 B16-F10 cells were grafted in the mammary fat pad of age-matched females 2-5 months old. Tumors were measured over time using a digital caliper. Tumors were then weighed post-euthanasia and analyzed via flow cytometry.

### Gene Silencing and Overexpression

GenMute transfection reagent (SignaGen) for primary macrophages was used for siRNA-mediated knockdowns. Effectene transfection reagent (Qiagen) was used for over-expression. Media was replaced 4 hours after transfection and all downstream analysis was carried out 48 hours after transfection.

### Flow Cytometry Staining and Analysis

For tissue samples, fresh tissues were collected from mice and digested in DMEM F12 supplemented by 2 mg/ml type II collagenase and 1% penicillin/streptomycin for 30-60 min at 37° C. while shaking. Cells were then passed through a 70 μm filter, washed with FACS, incubated with ACK lysis buffer followed by another wash with FACS Buffer. For surface staining, cells were fluorescently conjugated antibodies in FACS (PBS supplemented with 2% FBS and 1% penicillin-streptomycin) at a concentration of 1:50-1:100 for 30-60 minutes, followed by FACS washes and fixed in 4% formaldehyde if needed. Intracellular staining was carried out using BD Fix/perm reagents according to the manufacturer's instructions. FCS Express and companion software for the Thermo Attune instrument were used to capture and analyze data from flow cytometry.

T cell Migration Assay

A pre-optimized amount of BMDMs were seeded and primed with OVA and LPS. Pan T Cells were isolated from the spleen of OT-1 mice and co-cultured with pre-primed and pre-treated BMDMs for 72 hours. E0771-OVA cells were seeded on a 24-well plate. Post co-culture with BMDMs, T Cells were stained with cFSE, and a pre-determined amount of T Cells were added in the top chamber of a Boyden chamber with 3.0 μ pore size, T Cells were allowed to migrate for 10 hours. Cells were then lysed using Triton-X, fluorescence of migrated cells was measured using plate reader.

### T Cell Expansion Assay

A pre-optimized number of BMDMs was seeded followed by treatment or transfection. Pan T Cells were isolated from the spleen of wildtype C57BL6 mice using Untouched mouse T Cells Kit (Dynabeads), stained with cFSE (biolegend), and activated using antiCD3, antiCD28 antibodies (Biolegend). T Cells were co-cultured with pre-treated BMDMs for 72 hours, followed by staining of surface protein markers. Samples were analyzed using flow cytometry.

### T Cell Cytolysis Assay

A pre-optimized number of BMDMs was seeded and primed with OVA peptide and LPS. Pan T Cells were isolated from the spleen of OT-1 mice and co-cultured with pre-primed BMDMs for 72 hours. E0771 and E0771-OVA cells were labeled with cFSE and Cell Trace Far Red or Cell Trace Violet respectively and then plated in a 24-well plate. Subsequently, activated T Cells were counted and co-cultured with cancer cells at various ratios. 24 hours after co-culture, cytolytic activity was assessed by flow cytometry. The ratio between OVA-expressing cells and Parental cells was determined.

### Organoid Infiltration Assay

1.5% agarose was cast in 96 well u bottom ultra-low attachment plates. 4T1 or E0771 cells were pre-stained with HCS Nuclear Mask Blue (ThermoFIsher, Cat No: H10325) and Cell Mask Orange (Cat No: C10045), and layered on top of agarose gel in each well. Tumorspheres were cultured for 7 days, media was replaced every 3 days. On day 7, pre-treated BMDMs were stained with Cell Trace Red (ThermoFisher, Cat No: C34572) and resuspended and layered on top of organoids and allowed to infiltrate overnight. Next day, all media in the well was replaced with Live Cell Imaging solution (ThermoFisher, Cat No: A59688DJ), and tumor spheres were imaged using LSM900 Confocal Microscope.

### HUVEC Tube Formation Assay

HUVECs were procured from ATCC and cultured in Human Large Vessel Endothelial Cell Basal Medium (ThermoFisher) supplemented with Large Vessel Endothelial Supplement (LEVS) as per the manufacturer's instructions and 1% penicillin-streptomycin. 100 uL Geltrex (ThermoFisher) was evenly applied on a 24-well glass bottom plate and HUVEC cells were layered on top. After 4 hours, pre-treated BMDMs were co-cultured with HUVECs using a 0.4 μm Boyden chamber overnight. Post-co-culture HUVECs were stained with Calcein AM dye (ThermoFisher) and imaged using an LSM900 confocal microscope.

### Efferocytosis Assay

BMDMs were stained with Cell Tracer Red (ThermoFisher), treated, and seeded, at 80% confluency. Polymorphonuclear Neutrophils (PMNs) were isolated from the bone marrow of wildtype mice using Ly6G isolation beads (MiltenyiBiotec) as per manufacturer's instructions, stained with CFSE (Biolegend), and treated with staurosporine (Sigma Aldrich) for 4-6 hrs. Post-treatment, PMNs were washed with PBS twice and co-cultured overnight with BMDMs at a 1:2 BMDM:PMN ratio. Cells were analyzed using flow cytometry.

### Phagocytosis Assay

A pre-determined amount of BMDMs were seeded, and transfected or treated as needed. Phagocytosis Assay Kit was purchased from Cayman Chemicals and the experiment was carried out as per the manufacturer's instructions. Data was analyzed using Flow Cytometry.

### Human PBMC Analysis

Blood Samples from female volunteers was obtained, mononuclear cells were obtained by density gradient centrifugation using Lymphoprep medium (StemCell Cat No: 18060). T Cells were isolated using Pan T Cell isolation kit from Miltenyi as per manufacturer's instructions. T Cells once isolated were stored in Cytostor medium. Flowthrough from T Cell isolation was cultured in X-Vivo Medium supplemented with human m-csf (Peprotech) for 7 days to obtain mature human macrophages (hMDMs). T Cells were stained with cFSE and co-cultured with patient matched hMDMs for 72 hours followed by surface staining and flow cytometric analysis.

### RNA Sequencing and Data Analysis

Bulk RNA sequencing was conducted on an SP lane with 1×100 nt reads, generating more than 480 million reads.

DEG list was used to plot the heatmap and perform Gene Set Enrichment Analysis and Gene Ontology Analysis.

### ATAC Sequencing and Data Analysis

ATAC-seq fastq files were trimmed using Trim Galore (paired-end, quality score ≥30). Trimmed reads were aligned to the mouse genome (GRCm39) using Bowtie2, and the sorted and indexed BAM files created using Samtools. Peak calling was performed with MACS2 using the parameters -bdg-nomodel-g mm to identify accessible chromatin regions in each sample. To combine peaks across all samples, Bedtools merge was used to create a merged peak set, and coverage signal across all merged peaks was quantified with Bedtools coverage. Read counts were normalized to reads per million mapped reads (RPM), with the total number of mapped reads calculated using Samtools view (-c-F 256). Differential accessibility analysis was carried out using DESeq2 on three biological replicates of macrophage control and treatment samples, to identify differentially accessible regions. For motif enrichment analysis, peaks were divided into upregulated and downregulated groups based on the SWFC (significance weight fold change) score (−log 10(p_adj)*log 2(Fold Change)), with thresholds of −1 and 1 for upregulated and downregulated peaks, respectively. Genomic sequences of these peak groups were extracted using Bedtools getfasta, and motif enrichment was assessed using SEA (-thresh 10.0-align center) from the MEME Suite with the HOCOMOCOv11_full_mouse_mono motif database. Non-differential peaks (SFW between −1 and 1) were used as a control for motif enrichment analysis.

For differential ATAC-seq peak enrichment relative to gene expression signatures, transcript coordinates were obtained from BioMart. Genes were classified into upregulated, non-regulated, and downregulated categories based on their SWFC scores, using a threshold of ±0.8. Gene Ontology (GO) analysis was performed using GeneOntology.org to identify functional enrichments in these gene sets. To assess the enrichment of ATAC-seq peaks near upregulated and downregulated genes, the density of ATAC peaks was calculated for each gene set (upregulated and downregulated) across various window sizes ranging from 10 kb to 50 Mb, using the formula: density=(10\hat{ }8)*number of ATAC peaks/window size. The expected density for each set was calculated by permutation (×1000) against an equal-sized random gene set. This analysis was performed separately for both the upregulated and the downregulated ATAC peaks.

For differential expression enrichment of Gene Ontology (GO) terms, the mean SWFC score of genes within each GO term was calculated. The Z-score of enrichment for each GO term was determined by comparing the observed mean SWFC score to a null distribution generated through 1000 random permutations of similarly sized gene sets. To assess the nearby enrichment of upregulated ATAC-seq peaks in relation to GO terms, only the downregulated GO terms (SWFC<(−1)) were selected, as the downregulated genes showed closer proximity to the upregulated ATAC peaks in the above method. The density of ATAC peaks(upregulated) was calculated around the gene sets for each selected GO term using the formula: Density=(10\hat{ }6)×number of ATAC peaks/window size. The Z-score for each GO term was then calculated by comparing the observed ATAC peak density to a null distribution, which was created through 1000 random permutations of equal-sized random gene sets.

We used a variant of the SEAMoD tool for motif discovery. This tool uses an interpretable neural network model to relate cis-regulatory sequences (promoters and enhancers) to gene expression in varying cellular contexts. Motifs are learnt as convolutional filters that are parameters of the model. SEAMoD protocol was slightly modified to accommodate the data sparsity. Genes and the set of differentially expressed genes (DEGs) were split into three sets—training, validation and test. A DEG was assigned a value of 1 or −1 to indicate up and down regulation where as 0 if the expression didn't show a significant change in expression (these values make the model output). For each gene the set 4 nearest ATAC peaks (within 100 kbp from the transcription start site) in a cell type-condition pair were selected as the putative cis-regulatory modules (CRMs). All genes from the training set were used to train the model in the first round. Nearest accessible ATAC peak (nearest neighbor) for a gene in each cell type-condition pair were selected as the CRMs for the first round. This was followed by another round of model training using only those genes which were differentially expressed in at least one of the cell types and the CRMs were kept the same as the first round. Next the set of best CRMs (based on the model from the second training round) were identified (54). The model was trained for the third time using all genes from the training set and the set of best enhancers to fix the convolutional filters. Remaining trainable parameters were further fine-tuned by fourth round of model training (from the previous step) using only the DEGs while keeping the convolutional filters unchanged. This final model was then used to do all further analysis. In-silico knockdown was performed (Bhogale et al., SEAMoD: A fully interpretable neural network for cis-regulatory analysis of differentially expressed genes. BioRxiv. 2023 November 13:2023.11.09.565900. doi: 10.1101 2023.11.09.565900) to obtain the convolutional filters that were most responsible for the model's predictions. These filters were then converted into position weight matrices (PWMs) using the same protocol as described (54). The PWMs thus obtained were then compared to the a compendium of previously characterized PWMs in mouse using TOMTOM to obtain the final list of important PWMs and associated TFs. Motifs with TOMTOM q-value less than 1 were selected. The PWM logos were generated using ggseqlogo (Wagih et al., Bioinformatics 33, 3645-3647, 2017).

### Statistics

Data and variability was assessed for normality and handled as parametric, Ln transformed to normalize, or as non-parametric, as appropriate. Specific statistical tests used are denoted in the figure legends. A P-value of 0.5 was set as the threshold for significance. Asterisks (*) or different letters denote statistically significant differences. For example, a bar with “a” is different than a bar with “b” or “c”, but not different than a bar with “ab” or “ac”.

All publications, patents, and patent documents cited herein are incorporated by reference as though individually incorporated by reference. No limitations inconsistent with this disclosure are to be understood therefrom. The invention has been described with reference to various specific and preferred embodiments and techniques. However, many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

What is claimed is:

1. A method of cell therapy comprising the steps of
i) transforming a population of myeloid cells in vitro with a polynucleotide encoding an ABCA1 gene to form a population of engineered myeloid cells;
ii) expanding the population of engineered myeloid cells; and
iii) administering an effective amount of the expanded population of engineered myeloid cells to a subject having or suspected of having a cancer, thereby treating the cancer when present.

2. The method of claim 1, wherein the population of myeloid cells is derived from the subject having or suspected of having the cancer.

3. The method of claim 1, wherein the population of myeloid cells is derived from a donor subject that does not have cancer.

4. The method of claim 1, wherein the polynucleotide encodes a polypeptide that is at least about 85% identical to SEQ ID NO: 6.

5. The method of claim 1, wherein the polynucleotide encodes a polypeptide that is identical to SEQ ID NO: 6.

6. The method of claim 1, wherein the polynucleotide comprises a nucleotide sequence that is at least about 75% identical to SEQ ID NO: 5.

7. The method of claim 1, wherein the polynucleotide comprises a nucleotide sequence that is identical to SEQ ID NO: 5.

8. The method of claim 1, wherein the population of engineered myeloid cells overexpress the ABCA1 protein compared to the population of myeloid cells.

9. The method of claim 1, wherein the population of engineered myeloid cells comprises one or more of a hematopoietic stem cell, a hemocytoblast, a bone marrow cell, a bone marrow stem cell, a myeloid stem cell, a myeloid progenitors, a granulocyte, a monocyte, a macrophage, a dendritic cell, a myeloid derived suppressor cell, a monocytic myeloid derived suppressor cell, a granulocytic myeloid derived suppressor cell, and a mast cell.

10. A method of treating cancer in a subject comprising administering to the subject a therapeutically effective amount of an ApoA-I mimetic polypeptide, thereby treating the cancer, wherein the ApoA-I mimetic polypeptide comprises an amino acid sequence that is at least about 85% identical to:
(SEQ ID NO: 1)
DWLKAFYDKVAEKLKEAFPDWAKAAYDKAAEKAKEAA;
(SEQ ID NO: 2)
PVLDLFRELLNELLEALKQKLK;
(SEQ ID NO: 3)
DWFKAFYDKVAEKFKEAF;
or
(SEQ ID NO: 4)
^{D}D^{D}W^{D}F^{D}K^{D}A^{D}F^{D}Y^{D}D^{D}K^{D}V^{D}A^{D}E^{D}K^{D}E^{D}K^{D}E^{D}A^{D}F;
wherein superscript “D” indicates a D-amino acid, optionally, an acetyl moiety (Ac) is covalently bonded to an N-terminus nitrogen of the ApoA-I mimetic polypeptide, and optionally, an amine group is covalently bonded to a C-terminus carbon of the ApoA-I mimetic polypeptide.

11. The method of claim 10, wherein the ApoA-I mimetic polypeptide comprises the amino acid sequence:
(SEQ ID NO: 1)
Ac-DWLKAFYDKVAEKLKEAFPDWAKAAYDKAAEKAKEAA-NH₂;
(SEQ ID NO: 2)
Ac-PVLDLFRELLNELLEALKQKLK-N_{H2};
(SEQ ID NO: 3)
Ac-DWFKAFYDKVAEKFKEAF-NH₂;
and
(SEQ ID NO: 4)
AC-^{D}D^{D}W^{D}F^{D}K^{D}A^{D}F^{D}Y^{D}D^{D}K^{D}V^{D}A^{D}E^{D}K^{D}E^{D}K^{D}E^{D}A^{D}F-NH₂.

12. The method of claim 11, wherein the ApoA-I mimetic polypeptide is Ac-DWFKAFYDKVAEKFKEAF-NH₂ (SEQ ID NO: 3).

13. The method of claim 10, wherein the ApoA-I mimetic polypeptide comprises the amino acid sequence:
(SEQ ID NO: 1)
DWLKAFYDKVAEKLKEAFPDWAKAAYDKAAEKAKEAA;
(SEQ ID NO: 2)
PVLDLFRELLNELLEALKQKLK;
(SEQ ID NO: 3)
DWFKAFYDKVAEKFKEAF;
or
(SEQ ID NO: 4)
^{D}D^{D}W^{D}F^{D}K^{D}A^{D}F^{D}Y^{D}D^{D}K^{D}V^{D}A^{D}E^{D}K^{D}E^{D}K^{D}E^{D}A^{D}F.

14. The method of claim 10, wherein the ApoA-I mimetic polypeptide comprises the amino acid sequence:
(SEQ ID NO: 1)
Ac-DWLKAFYDKVAEKLKEAFPDWAKAAYDKAAEKAKEAA-NH₂;
(SEQ ID NO: 2)
Ac-PVLDLFRELLNELLEALKQKLK-N_{H2};
(SEQ ID NO: 3)
Ac-DWFKAFYDKVAEKFKEAF-NH₂;
and
(SEQ ID NO: 4)
AC-^{D}D^{D}W^{D}F^{D}K^{D}A^{D}F^{D}Y^{D}D^{D}K^{D}V^{D}A^{D}E^{D}K^{D}E^{D}K^{D}E^{D}A^{D}F-NH₂.

15. The method of claim 10, wherein the ApoA-I mimetic polypeptide is formulated as a composition comprising a pharmaceutically acceptable carrier, excipient, or diluent.

16. The method of claim 10, wherein the cancer is one or more of melanoma, kidney, hepatobiliary, head-neck squamous carcinoma, pancreatic, colon, bladder, glioblastoma, prostate, lung, and breast cancer.

17. The method of claim 15, wherein the cancer is the melanoma or the breast cancer.


SEQUENCE LISTING (6 sequences)

SEQ ID NO: 1 - protein, 37 aa, Synthetic construct
DWLKAFYDKVAEKLKEAFPDWAKAAYDKAAEKAKEAA

SEQ ID NO: 2 - protein, 22 aa, Synthetic construct
PVLDLFRELLNELLEALKQKLK

SEQ ID NO: 3 - protein, 18 aa, Synthetic construct
DWFKAFYDKVAEKFKEAF

SEQ ID NO: 4 - protein, 18 aa, Synthetic construct
  SITE 1: note = D-Aspartic Acid
  SITE 2: note = D-Tryptophan
  SITE 3: note = D-Phenylalanine
  SITE 4: note = D-Lysine
  SITE 5: note = D-Alanine
  SITE 6: note = D-Phenylalanine
  SITE 7: note = D-Tyrosine
  SITE 8: note = D-Aspartic Acid
  SITE 9: note = D-Lysine
  SITE 10: note = D-Valine
  SITE 11: note = D-Alanine
  SITE 12: note = D-Glutamic acid
  SITE 13: note = D-Lysine
  SITE 14: note = D-Phenylalanine
  SITE 15: note = D-Lysine
  SITE 16: note = D-Glutamic acid
  SITE 17: note = D-Alanine
  SITE 18: note = D-Phenylalanine
DWFKAFYDKVAEKFKEAF

SEQ ID NO: 5 - DNA, 6786 bp, Homo sapiens
  source 1..6786: mol_type = other DNA
atggcttgttggcctcagctgaggttgctgctgtggaagaacctcactttcagaagaagacaaacatgtcagctgctgctggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccaaaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacaaaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagagtgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatga

SEQ ID NO: 6 - protein, 2261 aa, Homo sapiens
MACWPQLRLLLWKNLTFRRRQTCQLLLEVAWPLFIFLILISVRLSYPPYEQHECHFPNKAMPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLDERKFWAGIVFTGITPGSIELPHHVKYKIRMDIDNVERTNKIKDGYWDPGPRADPFEDMRYVWGGFAYLQDVVEQAIIRVLTGTEKKTGVYMQQMPYPCYVDDIFLRVMSRSMPLFMTLAWIYSVAVIIKGIVYEKEARLKETMRIMGLDNSILWFSWFISSLIPLLVSAGLLVVILKLGNLLPYSDPSVVFVFLSVFAVVTILQCFLISTLFSRANLAAACGGIIYFTLYLPYVLCVAWQDYVGFTLKIFASLLSPVAFGFGCEYFALFEEQGIGVQWDNLFESPVEEDGFNLTTSVSMMLFDTFLYGVMTWYIEAVFPGQYGIPRPWYFPCTKSYWFGEESDEKSHPGSNQKRISEICMEEEPTHLKLGVSIQNLVKVYRDGMKVAVDGLALNFYEGQITSFLGHNGAGKTTTMSILTGLFPPTSGTAYILGKDIRSEMSTIRQNLGVCPQHNVLFDMLTVEEHIWFYARLKGLSEKHVKAEMEQMALDVGLPSSKLKSKTSQLSGGMQRKLSVALAFVGGSKVVILDEPTAGVDPYSRRGIWELLLKYRQGRTIILSTHHMDEADVLGDRIAIISHGKLCCVGSSLFLKNQLGTGYYLTLVKKDVESSLSSCRNSSSTVSYLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLRPFTEDDAADPNDSDIDPESRETDLLSGMDGKGSYQVKGWKLTQQQFVALLWKRLLIARRSRKGFFAQIVLPAVFVCIALVFSLIVPPFGKYPSLELQPWMYNEQYTFVSNDAPEDTGTLELLNALTKDPGFGTRCMEGNPIPDTPCQAGEEEWTTAPVPQTIMDLFQNGNWTMQNPSPACQCSSDKIKKMLPVCPPGAGGLPPPQRKQNTADILQDLTGRNISDYLVKTYVQIIAKSLKNKIWVNEFRYGGFSLGVSNTQALPPSQEVNDAIKQMKKHLKLAKDSSADRFLNSLGRFMTGLDTKNNVKVWFNNKGWHAISSFLNVINNAILRANLQKGENPSHYGITAFNHPLNLTKQQLSEVALMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTDNKLNNINDILKSVFLIFPHFCLGRGLIDMVKNQAMADALERFGENRFVSPLSWDLVGRNLFAMAVEGVVFFLITVLIQYRFFIRPRPVNAKLSPLNDEDEDVRRERQRILDGGGQNDILEIKELTKIYRRKRKPAVDRICVGIPPGECFGLLGVNGAGKSSTFKMLTGDTTVTRGDAFLNKNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVLKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV